SEQUENCE LISTING (6 sequences)

SEQ ID NO: 1 - protein, 1255 aa, Unknown
  SARS-CoV-1
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 222 aa, Unknown
  SARS-CoV-1
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 3 - protein, 1273 aa, Unknown
  SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 223 aa, Unknown
  SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 5 - protein, 1353 aa, Unknown
  MERS-CoV
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSTVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 6 - protein, 122 aa, Unknown
  MERS-CoV
ECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKC

(19) United States
(12) Patent Application Publication
(10) Pub. No.: US 2023/0201340 A1
(43) Pub. Date: Jun. 29, 2023

(54) ADJUVANTS FOR SEVERE ACUTE RESPIRATORY SYNDROME-RELATED CORONAVIRUS (SARS-COV) VACCINES

(71) Applicant: Children's Medical Center Corporation, Boston, MA (US)

(72) Inventors: David J. Dowling, Brighton, MA (US); Ofer Levy, Cambridge, MA (US); Francesco Borriello, Arlington, MA (US); Etsuro Nanishi, Boston, MA (US); Timothy O'Meara, Boston, MA (US); Yoshine Saito, Boston, MA (US); Simon D. van Haren, Wayland, MA (US)

(73) Assignee: Children's Medical Center Corporation, Boston, MA (US)

(21) Appl. No.: 17/928,255

(22) PCT Filed: May 28, 2021
(86) PCT No.: PCT/US2021/034919

§ 371 Date: Nov. 28, 2022

Related U.S. Application Data
(60) Provisional application No. 63/190,157, filed on May 18, 2021.
(60) Provisional application No. 63/032,422, filed on May 29, 2020.

(51) Int. Cl. A61K 39/39; A61K 39/215; A61P 31/14

(52) U.S. Cl. CPC A61K 39/39; A61K 39/215; A61P 31/14; C12N 2770/20034; A61K 2039/55561

(57) ABSTRACT

Provided herein are adjuvantation systems for use in Beta coronavirus (e.g., MERS-CoV, SARS-CoV-1, or SARS-CoV-2) vaccines and immunogenic compositions comprising the adjuvantation system and a Beta coronavirus antigen.

### RELATED APPLICATIONS

This application claims the benefit under 35 U.S.C. § 119(e) of U.S. Provisional Application Ser. No. 63/032,422 entitled “ADJUVANTS FOR SEVERE ACUTE RESPIRATORY SYNDROME-RELATED CORONAVIRUS (SARS-COV) VACCINES,” filed on May 29, 2020, and of U.S. Provisional Application Ser. No. 63/190,157 entitled “ADJUVANTS FOR SEVERE ACUTE RESPIRATORY SYNDROME-RELATED CORONAVIRUS (SARS-COV) VACCINES,” filed on May 18, 2021, the entire contents of each of which are incorporated herein by reference.

### GOVERNMENT SUPPORT

This invention was made with government support under 75N93019C00044 and HHSN272201400052C awarded by the National Institutes of Health and National Institutes of Health. The government has certain rights in the invention.

### BACKGROUND

Severe acute respiratory syndrome-related coronavirus (SARS-CoV) is a member of the genus Betacoronavirus and subgenus Sarbecoronavirus, and is a species of coronavirus that infects humans, bats and certain other mammals. It is an enveloped positive-sense single-stranded RNA virus that enters its host cell by binding to the angiotensin-converting enzyme 2 (ACE2) receptor

Two strains of the virus have caused outbreaks of severe respiratory diseases in humans: severe acute respiratory syndrome coronavirus (SARS-CoV or SARS-CoV-1), which caused the 2002-2004 outbreak of severe acute respiratory syndrome (SARS), and severe acute respiratory syndrome coronavirus 2 (SARS-CoV-2), which is causing the 2019-2020 pandemic of coronavirus disease 2019 (COVID-19). There are hundreds of other strains of SARS-CoV.

### SUMMARY

Discovery, development and implementation of safe and effective vaccines will be key to addressing the SARS-CoV-2 pandemic. Immunization of distinct vulnerable populations such as the elderly may result in sub-optimal responses, often requiring multiple booster doses and can be limited by waning immunity. Adjuvantation is a key approach to enhance vaccine-induced immunity. Adjuvants can enhance, prolong, and modulate immune responses to vaccinal antigens to maximize protective immunity, and may potentially enable effective immunization in vulnerable populations (e.g., in the very young and the elderly or for diseases lacking effective vaccines). Further, theoretical risk for SARS-CoV-2 vaccine-induced antibody disease enhancement (ADE) also needs to be addressed.

Some aspects of the present disclosure provide methods of inducing an immune response to a Beta coronavirus in a subject in need thereof, the method comprising administering to the subject a Beta coronavirus antigen and an adjuvantation system comprising a Pattern Recognition Receptor (PRR) agonist.

In some embodiments, the PRR agonist comprises a Toll-like Receptor (TLR) 3 agonist, a TLR4 agonist, a TLR9 agonist, or a Stimulator of Interferon Genes (STING) agonist. In some embodiments, the TLR3 agonist comprises polyinosinic:polycytidylic acid (Poly I:C). In some embodiments, the TLR4 agonist comprises phosphorylated hexa-acyl disaccharide (PHAD). In some embodiments, the TLR9 agonist comprises a CpG-containing oligodeoxynucleotide (CpG-ODN), such as a class A, class B, or class C CpG-ODN. In some embodiments, the class B CpG-ODN comprises CpG-ODN-1018. In some embodiments, the class C CpG-ODN comprises CpG-ODN-2395. In some embodiments, the STING agonist comprises 2′3′-cGAMP. In some embodiments, the adjuvantation system further comprises alum. In some embodiments, the PRR agonist is adsorbed into the alum.

In some embodiments, the Beta coronavirus is selected from Middle East Respiratory Syndrome coronavirus (MERS-CoV), Severe Acute Respiratory Syndrome (SARS)-associated coronavirus (SARS-CoV)-1, and SARS-CoV-2.

In some embodiments, the Beta coronavirus antigen comprises a Beta coronavirus protein or polypeptide. In some embodiments, the antigen comprises a nucleic acid encoding a Beta coronavirus protein or a polypeptide. In some embodiments, the nucleic acid is DNA or RNA. In some embodiments, the RNA is a messenger RNA (mRNA). In some embodiments, the Beta coronavirus protein or polypeptide comprises a Beta coronavirus spike protein or spike protein receptor binding domain. In some embodiments, the Beta coronavirus spike protein is a MERS-CoV spike protein, SARS-CoV-1 spike protein, or SARS-CoV-2 spike protein. In some embodiments, the antigen comprises a viral particle of MERS-CoV, SARS-CoV-1, or SARS-CoV-2. In some embodiments, the antigen comprises killed or inactivated MERS-CoV, SARS-CoV-1, or SARS-CoV-2. In some embodiments, the antigen comprises killed or live attenuated MERS-CoV, SARS-CoV-1, or SARS-CoV-2.

In some embodiments, the subject is human. In some embodiments, the subject is a human neonate, an infant, an adult, or an elderly. In some embodiments, the subject is a companion animal or a research animal. In some embodiments, the subject is immune-compromised, has chronic lung disease, asthma, cardiovascular disease, cancer, obesity, diabetes, chronic kidney disease, and/or liver disease.

In some embodiments, the Beta coronavirus antigen and the adjuvantation system are administered simultaneously. In some embodiments, the antigen and the adjuvantation system are administered separately. In some embodiments, the administering is done intramuscularly, intradermally, orally, intravenously, topically, intranasally, or sublingually. In some embodiments, the administration is prophylactic.

In some embodiments, the adjuvantation system enhances B cell immunity. In some embodiments, the adjuvantation system enhances the production of antigen-specific antibodies, compared to when the Beta coronavirus antigen is administered alone. In some embodiments, the antigen-specific antibodies are immunoglobulin G (IgG). In some embodiments, the antigen-specific antibodies are subclass 1 IgG (IgG1) or subclass 2 IgG (IgG2). In some embodiments, the antigen-specific antibodies are neutralizing antibodies against a variant of SARS-CoV-2. In some embodiments, the antigen-specific antibodies are neutralizing antibodies against wild-type SARS-CoV-2, B.1.1.7 SARS-CoV-2, or B.1.351 SARS-CoV-2. In some embodiments, the adjuvantation system enhances the cytokine production of peripheral blood mononuclear cells (PBMCs), compared to when the Beta coronavirus antigen is administered alone. In some embodiments, the PBMCs are antigen-specific T cells. In some embodiments, the adjuvantation system enhances the cytokine production of IL-2, IL-6, IL-10, TNF, IFNα, IFNγ, CCL3, CXCL8 and/or GM-CSF. In some embodiments, the adjuvantation system polarizes the innate immune response toward T follicular helper (Tfh) cell immunity. In some embodiments, the adjuvantation system polarizes the innate immune response toward T helper 1 (Th1) cell immunity. In some embodiments, the adjuvantation system enhances the inhibition of interaction between angiotensin-converting enzyme 2 (ACE2) and Beta coronavirus spike protein, compared to when the Beta coronavirus antigen is administered alone. In some embodiments, the adjuvantation system prolongs a protective effect in the subject against the Beta coronavirus antigen, compared to when the Beta coronavirus antigen is administered alone. In some embodiments, the adjuvantation system increases rate of an immune response, compared to when the Beta coronavirus antigen is administered alone. In some embodiments, the Beta coronavirus antigen produces a same level of immune response against the antigen at a lower dose in the presence of the adjuvantation system, compared to when the Beta coronavirus antigen is administered alone. In some embodiments, the likelihood of antibody disease enhancement (ADE) is reduced in the subject, compared to when the Beta coronavirus antigen is administered alone.

Other aspects of the present disclosure provide adjuvantation systems comprising a Pattern Recognition Receptor (PRR) agonist for use in inducing an immune response against a Beta coronavirus (e.g., MERS-CoV, SARS-CoV-1, or SARS-CoV-2) in a subject in need thereof.

In some aspects, an adjuvantation system comprising a Pattern Recognition Receptor (PRR) agonist and alum for use in inducing an immune response against a Beta coronavirus (e.g., MERS-CoV, SARS-CoV-1, or SARS-CoV-2) in a subject in need thereof.

In some embodiments, an adjuvantation system comprising a PRR agonist, whether in the presence or absence of alum, comprises a Toll-like Receptor (TLR) 3 agonist, a TLR4 agonist, a TLR9 agonist, or a Stimulator of Interferon Genes (STING) agonist. In some embodiments, the TLR3 agonist comprises polyinosinic:polycytidylic acid (Poly I:C). In some embodiments, the TLR4 agonist comprises phosphorylated hexa-acyl disaccharide (PHAD). In some embodiments, the TLR9 agonist comprises a CpG-containing oligodeoxynucleotide (CpG-ODN), such as a class A, class B, or class C CpG-ODN. In some embodiments, the class B CpG-ODN comprises CpG-ODN-1018. In some embodiments, the class C CpG-ODN comprises CpG-ODN-2395. In some embodiments, the STING agonist comprises 2′3′-cGAMP.

Also provided herein are immunogenic compositions comprising a Beta coronavirus antigen and an adjuvantation system comprising a Pattern Recognition Receptor (PRR) agonist. In some embodiments, the PRR agonist comprises a Toll-like Receptor (TLR) 3 agonist, a TLR4 agonist, a TLR9 agonist, or a Stimulator of Interferon Genes (STING) agonist. In some embodiments, the TLR3 agonist comprises polyinosinic:polycytidylic acid (Poly I:C). In some embodiments, the TLR4 agonist comprises phosphorylated hexa-acyl disaccharide (PHAD). In some embodiments, the TLR9 agonist comprises a CpG-containing oligodeoxynucleotide (CpG-ODN), such as a class A, class B, or class C CpG-ODN. In some embodiments, the class B CpG-ODN comprises CpG-ODN-1018. In some embodiments, the class C CpG-ODN comprises CpG-ODN-2395. In some embodiments, the STING agonist comprises 2′3′-cGAMP. In some embodiments, the adjuvantation system further comprises alum. In some embodiments, the PRR agonist is adsorbed into the alum.

In some embodiments, Beta coronavirus is selected from Middle East Respiratory Syndrome coronavirus (MERS-CoV), Severe Acute Respiratory Syndrome (SARS)-associated coronavirus (SARS-CoV)-1, and SARS-CoV-2. In some embodiments, the Beta coronavirus antigen comprises a Beta coronavirus protein or polypeptide. In some embodiments, the antigen comprises a nucleic acid encoding a Beta coronavirus protein or a polypeptide. In some embodiments, the nucleic acid is DNA or RNA. In some embodiments, the RNA is a messenger RNA (mRNA). In some embodiments, the Beta coronavirus protein or polypeptide comprises a Beta coronavirus spike protein or spike protein receptor binding domain. In some embodiments, the Beta coronavirus spike protein is a MERS-CoV spike protein, SARS-CoV-1 spike protein, or SARS-CoV-2 spike protein. In some embodiments, the antigen comprises a viral particle of MERS-CoV, SARS-CoV-1, or SARS-CoV-2. In some embodiments, the antigen comprises killed or inactivated MERS-CoV, SARS-CoV-1, or SARS-CoV-2. In some embodiments, the antigen comprises killed or live attenuated MERS-CoV, SARS-CoV-1, or SARS-CoV-2.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1: An adjuvantation system containing alum formulated with 2′3′-cGAMP enhances immunogenicity of SARS-CoV-1 spike protein RBD. 12 week old C57BL/6 mice were immunized (prime Day(D)0, boost D14) IM with PBS, SARS-CoV-1 RBD (10 μg) admixed with alum (100 μg) or alum (100 μg) formulated with 2′3′-cGAMP (10 μg). Anti-RBD IgG were quantified on D28 in plasma samples. Optical density (OD) values are depicted (median fold over background, BKG) of serial 4-fold dilutions starting from 1:100. N=4 mice per group. * and ** respectively indicate p<0.05 and p<0.01 measured by 2-way repeated measure ANOVA corrected for multiple comparisons on Log 10-transformed data. Light-shaded asterisk indicates statistical comparison vs. RBD+alum; dark-shaded asterisks indicate statistical comparison vs. PBS.

FIGS. 2A-2J: RBD formulated with AH:CpG induces robust production of anti-RBD neutralizing antibodies in young adult mice. Young, 3 months old BALB/c mice were immunized IM on Days 0 and 14 with 10 μg of monomeric SARS-CoV-2 RBD protein with indicated adjuvants. Each PRR agonist was administered alone or formulated with aluminum hydroxide (AH). Serum samples were collected on Day 28. Anti-RBD IgG (FIG. 2A), IgG1 (FIG. 2B), IgG2a (FIG. 2C), IgG2a/IgG1 ratio (FIG. 2D), hACE2/RBD inhibition rate (FIG. 2E), and anti-Spike IgG (FIG. 2F) were assessed. Serum samples were also collected on Day 210. Anti-RBD IgG (FIG. 2G), Anti-RBD IgG1 (FIG. 2H), Anti-RBD IgG2 (FIG. 2I), and hACE2/RBD inhibition rate (FIG. 2J) were assessed. N=10 per group. Data were combined from two individual experiments. Box and whisker represent minimum, first quartile, median, third quartile, and maximum value. Data were analyzed by two-way (FIGS. 2A-2C, FIGS. 2E-2F) (AH and PRR agonist) or one-way (FIG. 2D) ANOVAs followed by post-hoc Tukey's test for multiple comparisons. *P<0.05, **P<0.01, ***P<0.001, ****P<0.0001. Shaded asterisks, or where labels where provided, indicate statistical comparisons to RBD and AH-adjuvanted RBD groups according to shading. LLD, lower limit of detection.

FIGS. 3A-3M: AH:CpG adjuvant formulation elicits a robust anti-RBD response in aged mice. Aged, 14-month-old BALB/c mice were immunized IM on Days 0, 14, and 28 with 10 μg of monomeric SARS-CoV-2 RBD protein with indicated adjuvants. Each PRR agonist was formulated with aluminum hydroxide (AH). Serum samples were collected and analyzed on day 28 prior to the second boost (FIGS. 3A-3F), and day 42 (FIGS. 3G-M). Anti-RBD IgG (FIG. 3A, FIG. 3G), IgG1 (FIG. 3B, FIG. 3H), IgG2a (FIG. 3C, FIG. 3I), IgG2a/IgG1 ratio (FIG. 3D, FIG. 3J), hACE2/RBD inhibition rate (FIG. 3E, 3K), and neutralizing titer (FIG. 3F, FIG. 3L) was assessed. N=9-10 per group. Data were combined from two individual experiments and analyzed by one-way ANOVAs followed by post-hoc Tukey's test for multiple comparisons. FIG. 3M: Splenocytes were collected 2 weeks after the final immunization and stimulated with SARS-CoV-2 Spike peptide pool in the presence of anti-CD28 antibody (1 μg/ml). After 24 (for IL-2 and IL-4) and 96 (for IFNγ) hours, supernatants were harvested, and cytokine levels were measured by ELISA. N=4-5 per group. Data were log-transformed and analyzed by one-way ANOVAs followed by post-hoc Tukey's test for multiple comparisons. *P<0.05, **P<0.01, ***P<0.001, ****P<0.0001. Shaded asterisks, or labels where provided, indicate statistical comparisons to RBD and AH-adjuvanted RBD groups according to shading. Box and whisker represent minimum, first quartile, median, third quartile, and maximum value. LLD, lower limit of detection.

FIG. 4: Booster dose of AH:CpG formulation enhances hACE2/RBD inhibition in aged mice. Aged, 14-month-old BALB/c mice were immunized IM on Days 0, 14, and 28 with 10 μg of monomeric SARS-CoV-2 RBD protein with the indicated adjuvants. Serum samples were collected and analyzed on Day 28 prior to the 2nd boost, and Day 42. hACE2/RBD inhibition rate was assessed. N=9-10 animals per group. Data were combined from two individual experiments and analyzed by one-way ANOVA followed by post-hoc Tukey's test for multiple comparisons. Each dot represents individual results. Horizontal bars demonstrate mean plus SEM. ns: not significant, *P<0.05, **P<0.01. AH, aluminum hydroxide.

FIGS. 5A-5D: SARS-CoV-2 challenge model of young and aged mouse recapitulates human age-specific pathogenesis. Young (3-month-old) and aged (14-month-old) naïve BALB/c mice were challenged IN with mock (PBS), or 10², 10³, 10⁴, and 10⁵ PFU of mouse-adapted SARS-CoV-2 (MA10). Body weight change of (FIG. 5A) Young adult and (FIG. 5B) Aged mice were assessed daily up to 4 days post infection. Data represent mean and SEM with body weights only shown for surviving mice at each time-point. Data were analyzed by one-way ANOVA followed by Dunnett's test for comparisons between PBS group. FIG. 5C: Survival rate of aged mice. Data were analyzed by log-rank test in comparison to PBS group.

FIG. 5D: Viral titer in lung homogenates at 4-days post SARS-CoV-2 challenge (young: n=5 per group, aged: n=5 for 102; n=4 for 103; n=1 for 104; and n=0 for 105). Results represent mean±SEM. *P<0.05, **P<0.01, ***P<0.001, ****P<0.0001. Shaded asterisks, or labels where provided, indicate statistical comparisons to 10², 10³, 10⁴, and 10⁵ PFU MA10 groups according to shading FIG. 5E: Representative lung histological images at 4-days post challenge. Hematoxylin-eosin (H&E) staining is shown.

FIGS. 6A-6C: AH:CpG adjuvanted vaccine protects aged mice from SARS-CoV-2 challenge. Aged, 14-month-old BALB/c mice were immunized as in FIG. 2. On Day 70 (6 weeks post 2nd boost), mice were challenged IN with 103 PFU of mouse-adapted SARS-CoV-2 (MA10). FIG. 6A: Body weight changes were assessed daily up to 4 days post infection. Data represent mean and SEM. Data were analyzed by one-way ANOVA followed by Dunnett's Test for comparisons between PBS group. FIG. 6B: Viral titer in lung homogenates at 4-days post SARS-CoV-2 challenge. Results represent mean±SEM. Data were analyzed by one-way ANOVA followed by post-hoc Tukey's test for multiple comparisons. **P<0.01, ****P<0.0001. Shaded asterisks, or labels where provided, indicate comparisons to PBS, RBD, and RBD+aluminum hydroxide (AH) groups according to shading. LLD, lower limit of detection. FIG. 6C: Lung interstitial inflammation was evaluated and converted to a score of 0 to 4 with 0 being no inflammation and 4 being most severe. FIG. 6D: Representative lung histological images at 4-days post challenge. Hematoxylin-eosin (H&E) staining is shown.

FIGS. 7A-7D: AH:CpG-adjuvanted RBD vaccines and an authorized spike mRNA vaccine elicit comparable levels of neutralizing antibodies in aged mice. Aged, 14-month-old BALB/c mice were immunized IM on Days 0 and 14 with 10 μg of monomeric SARS-CoV-2 RBD protein with indicated adjuvants, or 1 μg of mRNA vaccine (BNT 162b2) as described in Methods. Serum samples were collected and analyzed on Day 28. Anti-RBD binding ELISA (FIG. 7A), anti-Spike binding ELISA (FIG. 7B), hACE2/RBD inhibition rate (FIG. 7C), and neutralizing titer (FIG. 7D) was assessed. N=9-10 per group. Data were combined from two individual experiments and analyzed by one-way ANOVAs followed by post-hoc Tukey's test for multiple comparisons. *P<0.05, **P<0.01, ***P<0.001, ****P<0.0001. Light-shaded asterisks indicate comparisons to PBS group. Box and whisker represent minimum, first quartile, median, third quartile, and maximum value. LLD, lower limit of detection. FIG. 7E: Pseudovirus neutralizing titers against wild-type or the B.1.17 or B.1.351 variants were assessed. N=5 per group. Values indicate geometric mean titer (GMT); each symbol represents one animal.

FIGS. 8A-8E: AH:CpG elicits comparable lymph node innate responses in young and aged mice. Young (3-month-old) and aged (14-month-old) mice were subcutaneously injected with aluminum hydroxide (AH), CpG, or AH:CpG. 24 hours later, draining LNs (dLNs) were collected, and RNA was extracted. FIG. 8A: Weights of dLNs were measured and expressed as fold over contralateral, PBS-injected LN. N=5 per group. # and ## respectively indicate P<0.05 and 0.01 when comparing each group against the value 1 (which represent the contralateral control sample expressed as fold). FIGS. 8B-8E: RNA isolated from dLNs was subjected to quantitative real-time PCR array comprised of 157 genes related to cytokines and chemokines, and type 1 IFN responses. N=4 animals per group. FIG. 8B: Principal component analysis demonstrated a marked separation by treatment and age. FIG. 8C: Unsupervised hierarchical clustering revealed major differences between treatments and highlighted the marked difference between AH and CpG-containing treatments. Each column represents gene categories and rows represent samples. FIG. 8D: Generalized linear model comparing treatment and age with each gene was performed. The top 4 significant genes (Ddx58, Ifit2, Isg15, Stat1) were selected and plotted with their relative expression values by age and treatment. Statistical analysis of the plots employed the Kruskal-Wallis test to compare mean differences across groups and Wilcoxon test to compare between ages. FIG. 8E: Enrichment analysis of differentially expressed genes using the blood transcriptional modules (see Li et. al, 2013; PMC: 24336226) was performed from the significant gene results after the generalized linear model by treatment. The top 20 modules are summarized per age.

FIGS. 9A-9D: AH:CpG synergistically enhances proinflammatory cytokine production from human adult and elderly PBMCs. Human PBMCs collected from young adult (FIG. 9A, FIG. 9C) and elderly individuals (FIG. 9B, FIG. 9D) were cultured in vitro for 24 hours with CpG alone (4, 10, 20, 40, and 100 μg/mL), aluminum hydroxide (AH) alone (8, 20, 40, 80, and 200 μg/mL), or combinations of each. Supernatants were collected for multiplexing bead array. N=6 per age group. FIGS. 9A-9B: Radar plot analysis of cytokines and chemokines are presented as a fold-change over the CpG alone group. FIGS. 9C-9D: Results represent mean±SEM. Unpaired Mann-Whitney test was applied at each concentration. Dark-shaded and light-shaded asterisks indicate comparisons of AH:CpG formulation to AH and CpG alone groups, respectively. *P<0.05, **P<0.01. Level of synergy was calculated using an adapted Loewe definition of additivity (D<1: synergy, D=1: additivity, D>1: antagonism).

### DETAILED DESCRIPTION OF CERTAIN EMBODIMENTS

Human immunity is crucial to both health and illness, playing key roles in multiple major diseases including infectious diseases, allergy and cancer. Infectious diseases are a leading cause of morbidity and mortality at the extremes of life. SARS-coronavirus-2 (SARS-CoV-2), the causal agent of COVID-19, first emerged in late 2019 in China. It has infected almost 160 million individuals and caused >3,300,000 deaths globally, especially in the elderly population. Discovery, development and implementation of safe and effective vaccines will be key to addressing the SARS-CoV-2 pandemic.

Immunization of distinct vulnerable populations such as the elderly may result in sub-optimal responses, often requiring multiple booster doses and can be limited by waning immunity. Adjuvantation is a key approach to enhance vaccine-induced immunity. Adjuvants can enhance, prolong, and modulate immune responses to vaccinal antigens to maximize protective immunity, and may potentially enable effective immunization in vulnerable populations (e.g., in the very young and the elderly or for diseases lacking effective vaccines). Further, theoretical risk for SARS-CoV-2 vaccine-induced antibody disease enhancement (ADE) also needs to be addressed.

Some aspects of the present disclosure provide immunogenic compositions (e.g., vaccine compositions) comprising a Beta coronavirus antigen and an adjuvantation system comprising a pattern recognition receptor (PRR) agonist. In some embodiments, the adjuvantation system further comprises alum (e.g., the PRR agonist is formulated with alum). In some embodiments, the PRR agonist is an agonist of a Toll-like receptor (TLR) or Stimulator of Interferon Genes (STING). In some embodiments, the PRR agonist is an agonist of TLR3, TLR4, or TLR9. In some embodiments, the TLR3 agonist is polyinosinic:polycytidylic acid (Poly I:C). In some embodiments, the TLR4 agonist is phosphorylated hexa-acyl disaccharide (PHAD). In some embodiments, the TLR9 agonist is a CpG-containing oligodeoxynucleotide (also termed a “CpG-ODN” herein), such as a class A, class B. or class C CpG-ODN. In some embodiments, the TLR9 agonist is CpG-ODN-1018 or CpG-ODN-2395. In some embodiments, the STING agonist is 2′3′-cGAMP (also termed “cGAMP” herein). In some embodiments, the PRR agonist (e.g., CpG-ODN-2395, cGAMP) is adsorbed in alum. The immunogenic composition (e.g., a vaccine composition) provided herein may be used in methods of inducing an immune response to an antigen in a subject in need thereof, the method comprising administering to the subject an effective amount of a Beta coronavirus antigen and an effective amount of the adjuvantation system (e.g., either comprising a PRR agonist alone, or comprising a PRR agonist and alum). In some embodiments, the immunogenic composition (e.g., vaccine composition) described herein may be used for inducing an immune response in a subject that is a newborn, an adult, or an elderly (e.g., a human subject older than 65 years old). In particular, the immunogenic composition (e.g., vaccine composition) described herein is effective for elderly immunization (i.e., for immunizing a human subject older than 65 years old).

“Beta coronavirus” is one of four genera (Alpha-, Beta-, Gamma-, and Delta-) of coronaviruses. It is in the subfamily Orthocoronavirinae in the family Coronaviridae, of the order Nidovirales. They are enveloped, positive-sense, single-stranded RNA viruses of zoonotic origin. Beta coronaviruses of the greatest clinical importance concerning humans SARS-CoV-1 (which causes severe acute respiratory syndrome, also referred to as SARS) and SARS-CoV-2 (which causes coronavirus disease 2019, also referred to as COVID-19), and MERS-CoV (which causes Middle East respiratory syndrome, also referred to as MERS).

“Pattern recognition receptors,” also referred to as PRRs herein, are protein components of the innate immune system that recognize pathogen-associated molecular patterns (PAMPs) and damage-associated molecular patterns (DAMPs). PRRs may occur in the cytoplasm of cells or on cellular membranes and are activated by interaction with specific PAMPs and/or DAMPs. PAMPs include molecules (e.g., proteins, nucleic acids, lipids) originating from organisms that are pathogenic or potentially pathogenic to a host, such as bacteria, viruses, or parasites. DAMPs include molecules that are associated with damage to a host (e.g. damaged proteins or nucleic acids). PRRs encompass a variety of different classes such as, but not limited to, Toll-like receptors, C-type lectin receptors, NOD-like receptors, and RIG-I-like receptors. Activation of PRRs typically results in a signaling cascade that results in transcriptional changes in a cell, such as the upregulated expression of one or more cytokines that enhance cellular immune responses. Cytokines produced through activation of PRRs can modulate responses in the same cell as the activated PRR or can be secreted to modulate immune responses in other cells. Cytokines produced by PRR activation can stimulate innate immunity and/or adaptive immunity.

“Toll-like receptors (TLRs)” are membrane-bound receptor proteins that detect PAMPs present in the extracellular environment and/or intracellular compartments, such as endosomes. TLRs may function as homodimers, or as heterodimers between two different TLR proteins. TLRs are critical for efficient detection and immunity against a broad range of PAMPs, including but not limited to those of viruses (e.g., viral RNA via TLR3, 7, and 8; viral DNA via TLR9), Gram-negative bacteria (e.g., lipopolysaccharide (LPS) via TLR4, flagellin via TLR5, DNA via TLR9), Gram-positive bacteria (e.g., lipoproteins via TLR1, 2, and 6; lipoteichoic acid (LTA) via TLR2, DNA via TLR9), fungi (e.g., zymosan and β-glycans via TLR2; DNA via TLR9), and protists (e.g., glycophosphatidylinositol (GPI) anchors via TLR2 and 4; DNA via TLR9). Humans express at least ten functional TLRs (TLR1, 2, 3, 4, 5, 6, 7, 8, 9, and 10), while mice express at least twelve (TLR1, 2, 3, 4, 5, 6, 7, 8, 9, 11, 12, 13). Following recognition of a PAMP, activated TLRs recruit adaptor proteins for signal transduction, such as MyD88 and TRIF, which activate downstream kinases that signal for the production of inflammatory cytokines.

“Stimulator of Interferon Genes (STING),” also known as MITA and MPYS, and encoded by TMEM173 gene, is a signaling molecule associated with the endoplasmic reticulum (ER) and is essential for controlling the transcription of numerous host defense genes, including type I interferons (IFNs) and pro-inflammatory cytokines, following the recognition of aberrant DNA species or cyclic dinucleotides (CDNs) in the cytosol of the cell.

A “PRR agonist” refers to a molecule that is capable of binding to and activating one or more PRRs. In some embodiments, a PRR agonist is a TLR agonist. In some embodiments, a PRR agonist is a STING agonist. A PRR agonist may be a naturally occurring agonist, a synthetic agonist, or a semi-synthetic agonist.

A “TLR agonist” refers to a molecule that can be recognized by a TLR and can activate a TLR signaling pathway. A TLR agonist may also be referred to as a “TLR ligand”. Natural TLR agonists broadly include nucleic acids, proteins, lipoproteins, lipids, and polysaccharides that are viral, bacterial, fungal, or protist in origin, as well as host molecules that become damaged, such as self-DNA that has leaked from the nucleus of the host cell.

In some embodiments, the TLR agonist for use in the immunogenic composition (e.g., vaccine composition) and methods described herein is an agonist of TLR3, TLR4, or TLR9. In some embodiments, the TLR agonist for use in the immunogenic composition (e.g., vaccine composition) and methods described herein is polyinosinic:polycytidylic acid, also termed “Poly I:C” herein. Poly I:C is a synthetic mimetic analog of viral double stranded DNA that induces the upregulation of type I interferon (IFN) inflammatory cytokines through activation of TLR3 (see Matsumoto M and Seya T. Adv Drug Deliv Rev. 2008 Apr. 29; 60(7):805-12, incorporated herein by reference).

In some embodiments, the TLR agonist for use in the immunogenic composition (e.g., vaccine composition) and methods described herein is phosphorylated hexa-acyl disaccharide (PHAD). PHAD is a synthetic analog of monophosphoryl lipid A (MPLA) that elicits production of inflammatory cytokines through activation of TLR4 (see Hernandez A et al., Crit Care Med. 2019 November; 47(11):e930-e938; Persing D H et al., Trends Microbiol. 2002; 10(10 Suppl):S32-7; and Baldridge J R et al., Methods. 1999 September; 19(1):103-7 incorporated by reference herein).

In some embodiments, the TLR agonist for use in the immunogenic composition (e.g., vaccine composition) and methods described herein is a CpG-containing oligodeoxynucleotide (CpG-ODN). CpG-ODNs are single-stranded synthetic DNA oligonucleotides that contain CpG dinucleotides. CpG dinucleotides (also known as “CpG sites” and “CpG motifs”) are frequent in bacterial genomes and are methylated to silence gene expression, however CpG dinucleotides are relatively uncommon in the genomes of many eukaryotes, including vertebrates. Unmethylated CpG dinucleotides potently induce production of inflammatory cytokines through activation of TLR9. CpG-ODNs are classified according to four distinct classes. Class A CpG-ODNs (also referred to as “Type D”) are characterized by a central self-complementing palindromic CpG-containing phosphodiester sequence and a phosphorothioate-modified 3′ poly-G tail. Class A CpG-ODNs are readily endocytosed and induce high IFNα production, but are otherwise generally weaker inducers of proinflammatory cytokine (e.g., IL-6) production compared to other classes. Class B CpG-ODNs (also referred to as “Type K”) are characterized by a completely phosphorothioate-modified backbone containing CpG dinucleotides, without palindromic sequences. Class B CpG-ODNs strongly induce production of proinflammatory cytokines (e.g., IL-6) through TLR9-dependent NF-1B signaling, but induce lower levels of IFN than class A. Class B CpG-ODNs also potently activate B cells. Class C CpG-ODNs share features of both class A and class B CpG-ODNs, having a completely phosphorothioate-modified backbone containing CpG dinucleotides and a palindromic sequence. Class C CpG-ODNs induce IFN similarly to Class A CpG-ODNs and proinflammatory cytokines (e.g., IL-6) similarly to class B CpG-ODNs, and are capable of activating B cells. In some embodiments, the CpG-ODN for use in the immunogenic composition (e.g., vaccine composition) and methods described herein is a class B CpG-ODN (e.g.). Class P CpG-ODNs are generally similar in structure and activity to those of class C but contain two separate palindromes. Information for CpG-ODNs and activities of classes thereof may be found, for instance, in Bode C et al. Expert Rev Vaccines. 2011 10(4): 499-511, which is incorporated by reference herein.

In some embodiments, the CpG-ODN for use in the immunogenic composition (e.g., vaccine composition) and methods described herein is a class B CpG-ODN. In some embodiments, the class B CpG-ODN for use in the immunogenic composition (e.g., vaccine composition) and methods described herein is CpG-ODN-1018, details for which may be found in Campbell J D, Methods Mol Biol. 2017; 1494:15-27, which is incorporated by reference herein. In some embodiments, the CpG-ODN for use in the immunogenic composition (e.g., vaccine composition) and methods described herein is a class C CpG-ODN.

In some embodiments, the class C CpG-ODN for use in the immunogenic composition (e.g., vaccine composition) and methods described herein is CpG-ODN-2395, details for which may be found in Li et al., Front Pharmacol. 2020 Feb. 6; 11:8; Malik A et al., Front Immunol. 2018 Mar. 20; 9:562; and Byadgi O et al., J Immunol Res. 2014; 2014:273284, which are incorporated by reference herein.

A “STING agonist” refers to a molecule that can be recognized by STING and can activate STING signaling pathway. A STING agonist may also be referred to as a “STING ligand”. Natural STING agonists include DNA that induce CDNs include the genome of invading pathogens, such as herpes simplex virus 1 (HSV1) or certain bacteria species. Self-DNA that has leaked from the nucleus of the host cell, perhaps following cell division or as a consequence of DNA damage, can also be potent activators of the STING pathway. Such DNA species may be responsible for causing various autoinflammatory diseases, such as systemic lupus erythematosus (SLE) or Aicardi-Goutières syndrome (AGS), and may influence inflammation-associated cancer. The commercially available STING agonist MK-1454 is a synthetic cyclic dinucleotide that has potent immunoactivating and antineoplastic activities.

In some embodiments, the STING agonist for use in the immunogenic composition (e.g., vaccine composition) and methods described herein is cyclic guanosine monophosphate (GMP)-adenosine monophosphate (AMP), also termed “cGAMP” herein. cGAMP has been shown to bind and activate STING (e.g., as described in Wang et al., Journal of Investigative Dermatology, Volume 136, Issue 11, November 2016, Pages 2183-2191, incorporated herein by reference). However, the effects of STING agonists (e.g., cGAMP) as vaccine adjuvants in certain subjects, such as newborn subjects, has not previously been investigated or demonstrated.

An “adjuvantation system” refers to a composition comprising one or more adjuvants. An “adjuvant” refers to a pharmacological or immunological agent that modifies the effect of other agents, for example, of an antigen in a vaccine. Adjuvants are typically included in vaccines to enhance the recipient subject's immune response to an antigen. The use of adjuvants allows the induction of a greater immune response in a subject with the same dose of antigen, or the induction of a similar level of immune response with a lower dose of injected antigen. Adjuvants are thought to function in several ways, including by increasing the surface area of antigen, prolonging the retention of the antigen in the body thus allowing time for the lymphoid system to have access to the antigen, slowing the release of antigen, targeting antigen to macrophages, activating macrophages, activating leukocytes such as antigen-presenting cells (e.g., monocytes, macrophages, and/or dendritic cells), or otherwise eliciting broad activation of the cells of the immune system see, e.g., H. S. Warren et al, Annu. Rev. immunol., 4:369 (1986), incorporated herein by reference. The ability of an adjuvant to induce and increase a specific type of immune response and the identification of that ability is thus a key factor in the selection of particular adjuvants for vaccine use against a particular pathogen. Adjuvants that are known to those of skill in the art, include, without limitation: aluminum salts (referred to herein as “alum”), liposomes, lipopolysaccharide (LPS) or derivatives such as monophosphoryl lipid A (MPLA) and glycopyranosyl lipid A (GLA), molecular cages for antigen, components of bacterial cell walls, endocytosed nucleic acids such as double-stranded RNA (dsRNA), single-stranded DNA (ssDNA), and unmethylated CpG dinucleotide-containing DNA. Typical adjuvants include water and oil emulsions, e.g., Freund's adjuvant and MF59, and chemical compounds such as aluminum hydroxide or alum. At present, currently licensed vaccines in the United States contain only a limited number of adjuvants, such as alum that enhances production of TH 2 cells and MPLA which activates innate immunity via Toll-like receptor 4 (TLR4). Many of the most effective adjuvants include bacteria or their products, e.g., microorganisms such as the attenuated strain of Mycobacterium bovis, Bacille Calmette-Guérin (BCG); microorganism components, e.g., alum-precipitated diphtheria toxoid, bacterial lipopolysaccharides (“endotoxins”) and their derivatives such as MPLA and GLA.

In some embodiments, the adjuvantation system of the present disclosure comprises a PRR agonist (e.g., CpG-ODN, cGAMP). In some embodiments, the adjuvantation system of the present disclosure comprises a PRR agonist (e.g., CpG-ODN, cGAMP) and aluminum salts (referred to herein as “alum”). In some embodiments, the alum is Alhydrogel® (InvivoGen, USA). In some embodiments, in an adjuvantation system comprising a a PRR agonist (e.g., CpG-ODN, cGAMP) and alum, the PRR agonist (e.g., CpG-ODN, cGAMP) is adsorbed into alum (e.g., as described in Jones et al., Journal of Biological Chemistry 280, 13406-13414, 2005, incorporated herein by reference).

Adjuvants or adjuvantation systems are used in immunogenic composition (e.g., the Beta coronavirus immunogenic composition (e.g., vaccine composition) described herein). The terms “vaccine composition” and “vaccine” are used interchangeably herein. An “immunogenic composition” is a composition that activates or enhances a subject's immune response to an antigen after the vaccine is administered to the subject. Vaccine compositions are a type of immunogenic compositions. In some embodiments, an immunogenic composition stimulates the subject's immune system to recognize the antigen (e.g., a Beta coronavirus antigen) as foreign, and enhances the subject's immune response if the subject is later exposed to the pathogen (e.g., Beta coronavirus), whether attenuated, inactivated, killed, or not. Vaccines may be prophylactic, for example, preventing or ameliorating a detrimental effect of a future exposure to a pathogen (e.g., Beta coronavirus), or therapeutic, for example, activating the subject's immune response to a pathogen after the subject has been exposed to the pathogen (e.g., Beta coronavirus). In some embodiments, an immunogenic composition (e.g., vaccine composition) is used to protect or treat an organism against a disease (e.g., MERS, SARS and/or COVID-19).

In some embodiments, the vaccine is a subunit vaccine (e.g., a recombinant subunit Beta coronavirus (e.g., MERS-CoV, SARS-CoV-1, or SARS-CoV-2) vaccine), an attenuated vaccine (e.g., containing an attenuated Beta coronavirus (e.g., MERS-CoV, SARS-CoV-1, or SARS-CoV-2) viral genome), a live vaccine (e.g., containing a live attenuated Beta coronavirus (e.g., MERS-CoV, SARS-CoV-1, or SARS-CoV-2)), or a conjugated vaccine (e.g., a vaccine containing an antigen (e.g., a Beta coronavirus (e.g., MERS-CoV, SARS-CoV-1, or SARS-CoV-2) antigen) that is not very immunogenic covalently attached to an antigen that is more immunogenic). One non-limiting example of a conjugated vaccine comprises a LPS attached to a strong protein antigen. In some embodiments, the vaccine comprises a killed/inactivated Beta coronavirus (e.g., MERS-CoV, SARS-CoV-1, or SARS-CoV-2). In some embodiments, the vaccine comprises a Beta coronavirus (e.g., MERS-CoV, SARS-CoV-1, or SARS-CoV-2) viral particle.

An “antigen” refers to an entity that is bound by an antibody or receptor, or an entity that induces the production of the antibody. In some embodiments, an antigen increases the production of antibodies that specifically bind the antigen. In some embodiments, an antigen comprises a protein or polypeptide. Such protein or peptide are referred to herein as “immunogenic polypeptide.” In some embodiments, the term “antigen” encompasses nucleic acids (e.g., DNA or RNA molecules) that encode immunogenic polypeptides. In some embodiments, the antigen is from a microbial pathogen. For example, the antigen may comprise parts (coats, capsules, cell walls, flagella, fimbriae, and toxins) of bacteria, viruses, fungi, and other microorganisms. For the purpose of the present disclosure, the antigen may comprise parts of a Beta coronavirus (e.g., MERS-CoV, SARS-CoV-1, or SARS-CoV-2).

In some embodiments, a protein or polypeptide antigen is a wild type protein or polypeptide. In some embodiments, a protein or polypeptide antigen is a polypeptide variant to a wild type protein or polypeptide. The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, polypeptide variants possess at least 50% identity to a native or reference sequence. In some embodiments, variants share at least 70%, at least 80%, at least 90%, at least 95%, or at least 99% identity with a native or reference sequence.

In some embodiments, a polypeptide variant comprises substitutions, insertions, deletions. In some embodiments, a polypeptide variant encompasses covalent variants and derivatives. The term “derivative” is used synonymously with the term “variant” but generally refers to a molecule that has been modified and/or changed in any way relative to a reference molecule or starting molecule.

In some embodiments, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

In some embodiments, the polypeptide variants comprises at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule. In some embodiments, the antigen is a polypeptide that includes 2, 3, 4, 5, 6, 7, 8, 9, 10, or more substitutions compared to a reference protein.

In some embodiments, the substitution is a conservative amino acids substitution. The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In some embodiments, protein fragments, functional protein domains, and homologous proteins are used as antigens in accordance with the present disclosure. For example, an antigen may comprise any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to a reference protein (e.g., a protein from a microbial pathogen) herein can be utilized in accordance with the disclosure.

In some embodiments, the antigen comprises more than one immunogenic proteins or polypeptides (e.g., 2, 3, 4, 5, 6, 7, 8, 9, 10, or more). In some embodiments, the more than one immunogenic proteins or polypeptides are derived from one protein (e.g., different fragments or one protein). In some embodiments, the more than one immunogenic proteins or polypeptides are derived from multiple proteins (e.g., from 2, 3, 4, 5, 6, 7, 8, 9, 10, or more proteins).

In some embodiments, the antigen comprises one or more immunogenic proteins, protein fragments or polypeptides that share at least 50%, at least 60%, at least 70%, at least 80%, at least 90%, at least 95%, or at least 99% identity to a reference sequence of a particular Beta coronavirus variant. In some embodiments, the variant is wild-type MERS-CoV, SARS-CoV-1, or SARS-CoV-2. In some embodiments, the variant is a Beta coronavirus variant that is not a wild-type variant. In some embodiments, the variant is a variant of SARS-CoV-2 that is not wild-type SARS-CoV-2, such as, but not limited to, B.1.1.7, B.1.351, P.1, B.1.427, B.1.429, B.1.526, B.1.526.1, B.1.525, P.2, B.1.617, B.1.617.1, B.1.617.2, or B.1.617.3 SARS-CoV-2.

In some embodiments, the antigen comprises a nucleic acid encoding an immunogenic protein or polypeptide. In some embodiments, the antigen comprises an immunogenic protein or polypeptide and a nucleic acid encoding the immunogenic protein or polypeptide. The term “nucleic acid” or “polynucleotide,” in its broadest sense, includes any compound and/or substance that comprises a polymer of nucleotides. Nucleic acids encoding immunogenic proteins or polypeptides typically comprise an open reading frame (ORF), and one or more regulatory sequences. Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, the nucleic acid encoding the immunogenic polypeptide is a DNA (e.g., an expression vector for an immunogenic protein or polypeptide). In some embodiments, the nucleic acid encoding the immunogenic polypeptide is a RNA (e.g., a messenger RNA). A “messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ, or ex vivo. The basic components of an mRNA molecule typically include at least one coding region, a 5′ untranslated region (UTR), a 3′ UTR, a 5′ cap and a poly-A tail.

In some embodiments, the coding region of the nucleic acid (e.g., DNA or RNA) encoding an immunogenic polypeptide is codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding an immunogenic protein or polypeptide). In some embodiments, a codon optimized sequence shares less than 90% sequence identity to a naturally-occurring or wild-type sequence (e.g., a naturally-occurring or wild-type mRNA sequence encoding an immunogenic protein or polypeptide). In some embodiments, a codon optimized sequence shares less than 85% sequence identity to a naturally-occurring or wild-type sequence (e.g., a naturally-occurring or wild-type mRNA sequence encoding an immunogenic protein or polypeptide). In some embodiments, a codon optimized sequence shares less than 80% sequence identity to a naturally-occurring or wild-type sequence (e.g., a naturally-occurring or wild-type mRNA sequence encoding an immunogenic protein or polypeptide). In some embodiments, a codon optimized sequence shares less than 75% sequence identity to a naturally-occurring or wild-type sequence (e.g., a naturally-occurring or wild-type mRNA sequence encoding an immunogenic protein or polypeptide).

In some embodiments, the nucleic acid encoding an immunogenic protein or polypeptide comprises one or more chemical modifications. The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population.

In some embodiments, the nucleic acids (e.g., DNA or RNA) comprise various (more than one) different modifications. In some embodiments, a particular region of a nucleic acid (e.g., DNA or RNA) contains one, two or more (optionally different) nucleoside or nucleotide modifications. In some embodiments, a modified nucleic acid (e.g., DNA or RNA), introduced to a cell or organism, exhibits reduced degradation in the cell or organism, respectively, relative to an unmodified nucleic acid. In some embodiments, a modified nucleic acid (e.g., DNA or RNA), introduced into a cell or organism, may exhibit reduced immunogenicity in the cell or organism, respectively (e.g., a reduced innate response).

Modified nucleic acid (e.g., DNA or RNA) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone). Modified nucleic acid (e.g., DNA or RNA), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

In some embodiments, a chemically modified nucleic acid comprises one or more modified nucleosides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

In some embodiments, a modified nucleobase is a modified uridine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine. In some embodiments, a modified nucleobase is a modified uridine. Exemplary nucleobases and In some embodiments, a modified nucleobase is a modified cytosine. nucleosides having a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

In some embodiments, the antigen comprises a viral protein and/or a nucleic acid encoding a viral protein (e.g., a viral structural or non-structural protein). In some embodiments, the antigen comprises a nucleic acid encoding the viral genome. In some embodiments, the viral genome is modified to produce a modified virus that is attenuated.

Polypeptide or polynucleotide molecules of the present disclosure may share a certain degree of sequence similarity or identity with reference molecules (e.g., reference polypeptides or reference polynucleotides), for example, wild-type molecules. The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197.) A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g., between nucleic acid molecules (e.g., DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In some embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

In some embodiments, the immunogenic compositions (e.g., vaccine compositions) described herein induce an immune response to a Beta coronavirus antigen (e.g., an antigen from any Beta coronavirus such as an antigen from MERS-CoV, SARS-CoV-1, or SARS-CoV-2) or to a Beta coronavirus (any Beta coronavirus species such as MERS-CoV, SARS-CoV-1, or SARS-CoV-2). In some embodiments, Beta coronavirus antigen used in the immunogenic composition described herein comprises an antigen (e.g., a protein or a nucleic acid) from MERS-CoV. In some embodiments, Beta coronavirus antigen used in the immunogenic composition described herein comprises an antigen (e.g., a protein or a nucleic acid) from SARS-CoV-1. In some embodiments, Beta coronavirus antigen used in the immunogenic composition described herein comprises an antigen (e.g., a protein or a nucleic acid) from SARS-CoV-2. In some embodiments, the immunogenic composition (e.g., vaccine composition) induces an immune response against MERS-CoV, SARS-CoV-1 and/or SARS-CoV-2. Heterologous immunity is contemplated herein. Heterologous immunity refers to phenomenon by which antigen-specific response that were generated against one pathogen are reactivated in response to a second pathogen. For example, the immunogenic composition (e.g., vaccine composition) may comprises a SARS-CoV-1 antigen and induces immune response to both SARS-CoV-1 and SARS-CoV-2. Similarly, the immunogenic composition (e.g., vaccine composition) may comprises a SARS-CoV-2 antigen and induces immune response to both SARS-CoV-1 and SARS-CoV-2.

In some embodiments, the Beta coronavirus antigen used in the immunogenic composition (e.g., vaccine composition) described herein comprises a Beta coronavirus (e.g., MERS-CoV, SARS-CoV-1, or SARS-CoV-2) protein or polypeptide, or an immunogenic fragment or variant thereof. In some embodiments, the Beta coronavirus antigen used in the immunogenic composition (e.g., vaccine composition) described herein comprises a nucleic acid (e.g., DNA or RNA such as mRNA) encoding a Beta coronavirus (e.g., MERS-CoV, SARS-CoV-1, or SARS-CoV-2) protein or polypeptide, or an immunogenic fragment or variant thereof.

In some embodiments, the Beta coronavirus antigen in the immunogenic composition (e.g., vaccine composition) described herein comprises a MERS-CoV spike protein, or an immunogenic fragment thereof (e.g., the receptor binding domain of the spike protein). In some embodiments, the Beta coronavirus antigen in the immunogenic composition (e.g., vaccine composition) described herein comprises a nucleic acid (e.g., DNA or RNA such as mRNA) MERS-CoV spike protein, or an immunogenic fragment thereof (e.g., the receptor binding domain of the spike protein).

In some embodiments, the Beta coronavirus antigen in the immunogenic composition (e.g., vaccine composition) described herein comprises a SARS-CoV-1 spike protein, or an immunogenic fragment thereof (e.g., the receptor binding domain of the spike protein). In some embodiments, the Beta coronavirus antigen in the immunogenic composition (e.g., vaccine composition) described herein comprises a nucleic acid (e.g., DNA or RNA such as mRNA) SARS-CoV-1 spike protein, or an immunogenic fragment thereof (e.g., the receptor binding domain of the spike protein).

In some embodiments, the Beta coronavirus antigen in the immunogenic composition (e.g., vaccine composition) described herein comprises a SARS-CoV-2 spike protein, or an immunogenic fragment thereof (e.g., the receptor binding domain of the spike protein). In some embodiments, the Beta coronavirus antigen in the immunogenic composition (e.g., vaccine composition) described herein comprises a nucleic acid (e.g., DNA or RNA such as mRNA) SARS-CoV-2 spike protein, or an immunogenic fragment thereof (e.g., the receptor binding domain of the spike protein).

Amino acid and nucleic acid (DNA or RNA) sequences of examples of Beta coronavirus antigen in the immunogenic composition (e.g., vaccine composition) described herein are provided in Table 1.

TABLE 1
Beta coronavirus antigens

| Antigen | Amino Acid Sequence |
|---|---|
| SARS-CoV-1 | MFIFLLFLTLTSGSDLDRCTTFDDVQAPNYTQHTSSMRGVYYPDEIFRSDTLYLTQDLFLPFY |
| Spike Protein | SNVTGFHTINHTFGNPVIPFKDGIYFAATEKSNVVRGWVFGSTMNNKSQSVIIINNSTNVVIR |
| (SEQ ID NO: 1) | ACNFELCDNPFFAVSKPMGTQTHTMIFDNAFNCTFEYISDAFSLDVSEKSGNFKHLREFVFKN |
| | KDGFLYVYKGYQPIDVVRDLPSGFNTLKPIFKLPLGINITNFRAILTAFSPAQDIWGTSAAAY |
| | FVGYLKPTTFMLKYDENGTITDAVDCSQNPLAELKCSVKSFEIDKGIYQTSNFRWPSGDWRFP |
| | NITNLCPFGEVFNATKFPSVYAWERKKISNCVADYSVLYNSTFFSTFKCYGVSATKLNDLCFS |
| | NVYADSFVVKGDDVRQIAPGQTGVIADYNYKLPDDFMGCVLAWNTRNIDATSTGNYNYKYRYL |
| | RHGKLRPFERDISNVPFSPDGKPCTPPALNCYWPLNDYGFYTTTGIGYQPYRVVVLSFELLNA |
| | PATVCGPKLSTDLIKNQCVNFNFNGLTGTGVLTPSSKRFQPFQQFGRDVSDFTDSVRDPKTSE |
| | ILDISPCSFGGVSVITPGTNASSEVAVLYQDVNCTDVSTAIHADQLTPAWRIYSTGNNVFQTQ |
| | AGCLIGAEHVDTSYECDIPIGAGICASYHTVSLLRSTSQKSIVAYTMSLGADSSIAYSNNTIA |
| | IPTNFSISITTEVMPVSMAKTSVDCNMYICGDSTECANLLLQYGSFCTQLNRALSGIAAEQDR |
| | NTREVFAQVKQMYKTPTLKYFGGFNFSQILPDPLKPTKRSFIEDLLFNKVTLADAGFMKQYGE |
| | CLGDINARDLICAQKFNGLTVLPPLLTDDMIAAYTAALVSGTATAGWTFGAGAALQIPFAMQM |
| | AYRFNGIGVTQNVLYENQKQIANQFNKAISQIQESLTTTSTALGKLQDWNQNAQALNTLVKQL |
| | SSNFGAISSVLNDILSRLDKVEAEVQIDRLITGRLQSLQTYVTQQLIRAAEIRASANLAATKM |
| | SECVLGQSKRVDFCGKGYHLMSFPQAAPHGVVFLHVTYVPSQERNFTTAPAICHEGKAYFPRE |
| | GVFVFNGTSWFITQRNFFSPQIITTDNTFVSGNCDVVIGIINNTVYDPLQPELDSFKEELDKY |
| | FKNHTSPDVDLGDISGINASVVNIQKEIDRLNEVAKNLNESLIDLQELGKYEQYIKWPWYVWL |
| | GFIAGLIAIVMVTILLCCMTSCCSCLKGACSCGSCCKFDEDDSEPVLKGVKLHYT |
| SARS-CoV-1 | RVVPSGDVVRFPNITNLCPFGEVFNATKFPSVYAWERKKISNCVADYSVLYNSTFFSTFKCYG |
| Spike Protein | VSATKLNDLCFSNVYADSFVVKGDDVRQIAPGQTGVIADYNYKLPDDFMGCVLAWNTRNIDAT |
| receptor | STGNYNYKYRYLRHGKLRPFERDISNVPFSPDGKPCTPPALNCYWPLNDYGFYTTTGIGYQPY |
| binding domain | RVVVLSFELLNAPATVCGPKLSTDLIKNQCVNF |
| (SEQ ID NO: 2) | |
| SARS-CoV-2 | MFVFLVLLPLVSSQCVNLTTRTQLPPAYTNSFTRGVYYPDKVFRSSVLHSTQDLFLPFFSNVT |
| Spike Protein | WFHAIHVSGTNGTKRFDNPVLPFNDGVYFASTEKSNIIRGWIFGTTLDSKTQSLLIVNNATNV |
| (SEQ ID NO: 3) | VIKVCEFQFCNDPFLGVYYHKNNKSWMESEFRVYSSANNCTFEYVSQPFLMDLEGKQGNFKNL |
| | REFVFKNIDGYFKIYSKHTPINLVRDLPQGFSALEPLVDLPIGINITRFQTLLALHRSYLTPG |
| | DSSSGWTAGAAAYYVGYLQPRTFLLKYNENGTITDAVDCALDPLSETKCTLKSFTVEKGIYQT |
| | SNFRVQPTESIVRFPNITNLCPFGEVFNATRFASVYAWNRKRISNCVADYSVLYNSASFSTFK |
| | CYGVSPTKLNDLCFTNVYADSFVIRGDEVRQIAPGQTGKIADYNYKLPDDFTGCVIAWNSNNL |
| | DSKVGGNYNYLYRLFRKSNLKPFERDISTEIYQAGSTPCNGVEGFNCYFPLQSYGFQPTNGVG |
| | YQPYRVVVLSFELLHAPATVCGPKKSTNLVKNKCVNFNFNGLTGTGVLTESNKKFLPFQQFGR |
| | DIADTTDAVRDPQTLEILDITPCSFGGVSVITPGTNTSNQVAVLYQDVNCTEVPVAIHADQLT |
| | PTWRVYSTGSNVFQTRAGCLIGAEHVNNSYECDIPIGAGICASYQTQTNSPRRARSVASQSII |
| | AYTMSLGAENSVAYSNNSIAIPTNFTISVTTEILPVSMTKTSVDCTMYICGDSTECSNLLLQY |
| | GSFCTQLNRALTGIAVEQDKNTQEVFAQVKQIYKTPPIKDFGGFNFSQILPDPSKPSKRSFIE |
| | DLLFNKVTLADAGFIKQYGDCLGDIAARDLICAQKFNGLTVLPPLLTDEMIAQYTSALLAGTI |
| | TSGWTFGAGAALQIPFAMQMAYRFNGIGVTQNVLYENQKLIANQFNSAIGKIQDSLSSTASAL |
| | GKLQDWNQNAQALNTLVKQLSSNFGAISSVLNDILSRLDKVEAEVQIDRLITGRLQSLQTYVT |
| | QQLIRAAEIRASANLAATKMSECVLGQSKRVDFCGKGYHLMSFPQSAPHGVVFLHVTYVPAQE |
| | KNFTTAPAICHDGKAHFPREGVFVSNGTHWFVTQRNFYEPQIITTDNTFVSGNCDVVIGIVNN |
| | TVYDPLQPELDSFKEELDKYFKNHTSPDVDLGDISGINASVVNIQKEIDRLNEVAKNLNESLI |
| | DLQELGKYEQYIKWPWYIWLGFIAGLIAIVMVTIMLCCMTSCCSCLKGCCSCGSCCKFDEDDS |
| | EPVLKGVKLHYT |
| SARS-CoV-2 | RVQPTESIVRFPNITNLCPFGEVFNATRFASVYAWNRKRISNCVADYSVLYNSASFSTFKCYG |
| Spike Protein | VSPTKLNDLCFTNVYADSFVIRGDEVRQIAPGQTGKIADYNYKLPDDFTGCVIAWNSNNLDSK |
| receptor | VGGNYNYLYRLFRKSNLKPFERDISTEIYQAGSTPCNGVEGFNCYFPLQSYGFQPTNGVGYQP |
| binding domain | YRVVVLSFELLHAPATVCGPKKSTNLVKNKCVNF |
| (SEQ ID NO: 4) | |
| MERS Spike | MIHSVFLLMFLLTPTESYVDVGPDSVKSACIEVDIQQTFFDKTWPRPIDVSKADGIIYPQGRT |
| protein | YSNITITYQGLFPYQGDHGDMYVYSAGHATGTTPQKLFVANYSQDVKQFANGFVVRIGAAANS |
| (SEQ ID NO: 5) | TGTVIISPSTSATIRKIYPAFMLGSSVGNFSDGKMGRFFNHTLVLLPDGCGTLLRAFYCILEP |
| | RSGNHCPAGNSYTSFATYHTPATDCSDGNYNRNASLNSFKEYFNLRNCTFMYTYNITEDEILE |
| | WFGITQTAQGVHLFSSRYVDLYGGNMFQFATLPVYDTIKYYSIIPHSIRSIQSDRKAWAAFYV |
| | YKLQPLTFLLDFSVDGYIRRAIDCGFNDLSQLHCSYESFDVESGVYSVSSFEAKPSGSVVEQA |
| | EGVECDFSPLLSGTPPQVYNFKRLVFTNCNYNLTKLLSLFSVNDFTCSQISPAAIASNCYSSL |
| | ILDYFSYPLSMKSDLSVSSAGPISQFNYKQSFSNPTCLILATVPHNLTTITKPLKYSYINKCS |
| | RLLSDDRTEVPQLVNANQYSPCVSTVPSTVWEDGDYYRKQLSPLEGGGWLVASGSTVAMTEQL |
| | QMGFGITVQYGTDTNSVCPKLEFANDTKIASQLGNCVEYSLYGVSGRGVFQNCTAVGVRQQRF |
| | VYDAYQNLVGYYSDDGNYYCLRACVSVPVSVIYDKETKTHATLFGSVACEHISSTMSQYSRST |
| | RSMLKRRDSTYGPLQTPVGCVLGLVNSSLFVEDCKLPLGQSLCALPDTPSTLTPRSVRSVPGE |
| | MRLASIAFNHPIQVDQLNSSYFKLSIPTNFSFGVTQEYIQTTIQKVTVDCKQYVCNGFQKCEQ |
| | LLREYGQFCSKINQALHGANLRQDDSVRNLFASVKSSQSSPIIPGFGGDFNLTLLEPVSISTG |
| | SRSARSAIEDLLFDKVTIADPGYMQGYDDCMQQGPASARDLICAQYVAGYKVLPPLMDVNMEA |
| | AYTSSLLGSIAGVGWTAGLSSFAAIPFAQSIFYRLNGVGITQQVLSENQKLIANKFNQALGAM |
| | QTGFTTTNEAFRKVQDAVNNNAQALSKLASELSNTFGAISASIGDIIQRLDVLEQDAQIDRLI |
| | NGRLTTLNAFVAQQLVRSESAALSAQLAKDKVNECVKAQSKRSGFCGQGTHIVSFVVNAPNGL |
| | YFMHVGYYPSNHIEVVSAYGLCDAANPTNCIAPVNGYFIKTNNTRIVDEWSYTGSSFYAPEPI |
| | TSLNTKYVAPQVTYQNISTNLPPPLLGNSTGIDFQDELDEFFKNVSTSIPNFGSLTQINTTLL |
| | DLTYEMLSLQQVVKALNESYIDLKELGNYTYYNKWPWYIWLGFIAGLVALALCVFFILCCTGC |
| | GTNCMGKLKCNRCCDRYEEYDLEPHKVHVH |
| MERS Spike | ECDFSPLLSGTPPQVYNFKRLVFTNCNYNLTKLLSLFSVNDFTCSQISPAAIASNCYSSLILD |
| protein | YFSYPLSMKSDLSVSSAGPISQFNYKQSFSNPTCLILATVPHNLTTITKPLKYSYINKC |
| receptor | |
| binding domain | |
| (SEQ ID NO: 6) | |

In some embodiments, the Beta coronavirus antigen in the immunogenic composition (e.g., vaccine composition) described herein comprises a protein having an amino acid sequence that is at least 70% (e.g., at least 70%, at least 75%, at least 80%, at least 85%, at least 90%, at least 95%, or at least 99%) identical to any one of SEQ ID NOs: 1-6. In some embodiments, the Beta coronavirus antigen in the immunogenic composition (e.g., vaccine composition) described herein comprises a protein having an amino acid sequence that is 70%, 75%, 80%, 85%, 90%, 95%, or 99% identical to any one of SEQ ID NOs: 1-6. In some embodiments, the Beta coronavirus antigen in the immunogenic composition (e.g., vaccine composition) described herein comprises a protein comprising the amino acid sequence of any one of SEQ ID NO: 1-6.

In some embodiments, the Beta coronavirus antigen in the immunogenic composition (e.g., vaccine composition) described herein comprises a nucleic acid (e.g., DNA or RNA such as mRNA) encoding a protein having an amino acid sequence that is at least 70% (e.g., at least 70%, at least 75%, at least 80%, at least 85%, at least 90%, at least 95%, or at least 99%) identical to any one of SEQ ID NOs: 1-6. In some embodiments, the Beta coronavirus antigen in the immunogenic composition (e.g., vaccine composition) described herein comprises a nucleic acid (e.g., DNA or RNA such as mRNA) encoding a protein having an amino acid sequence that is 70%, 75%, 80%, 85%, 90%, 95%, or 99% identical to any one of SEQ ID NOs: 1-6. In some embodiments, the Beta coronavirus antigen in the immunogenic composition (e.g., vaccine composition) described herein comprises a nucleic acid (e.g., DNA or RNA such as mRNA) encoding a protein comprising the amino acid sequence of any one of SEQ ID NO: 1-6.

In some embodiments, the immunogenic composition (e.g., vaccine composition) described herein are formulated for administration to a subject. In some embodiments, the immunogenic composition (e.g., vaccine composition) is formulated or administered in combination with one or more pharmaceutically-acceptable excipients. In some embodiments, immunogenic compositions (e.g., vaccine composition) comprise at least one additional active substances, such as, for example, a therapeutically-active substance, a prophylactically-active substance, or a combination of both. Immunogenic compositions (e.g., vaccine composition) may be sterile, pyrogen-free or both sterile and pyrogen-free. General considerations in the formulation and/or manufacture of pharmaceutical agents, such as immunogenic compositions (e.g., vaccine composition), may be found, for example, in Remington: The Science and Practice of Pharmacy 21st ed., Lippincott Williams & Wilkins, 2005 (incorporated herein by reference in its entirety).

Formulations of the immunogenic composition (e.g., vaccine composition) described herein may be prepared by any method known or hereafter developed in the art of pharmacology. In general, such preparatory methods include the step of bringing the antigen and/or the adjuvant (e.g., PRR agonist alone or PRR agonist and alum) into association with an excipient and/or one or more other accessory ingredients, and then, if necessary and/or desirable, dividing, shaping and/or packaging the product into a desired single- or multi-dose unit.

Relative amounts of the antigen, the adjuvant, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, the immunogenic composition (e.g., vaccine composition) described herein are formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA vaccines (e.g., for transplantation into a subject), hyaluronidase, nanoparticle mimics and combinations thereof.

In some embodiments, the immunogenic composition (e.g., vaccine composition) is formulated in an aqueous solution. In some embodiments, the immunogenic composition (e.g., vaccine composition) is formulated in a nanoparticle. In some embodiments, the immunogenic composition (e.g., vaccine composition) is formulated in a lipid nanoparticle. In some embodiments, the immunogenic composition (e.g., vaccine composition) is formulated in a lipid-polycation complex, referred to as a lipid nanoparticle. The formation of the lipid nanoparticle may be accomplished by methods known in the art and/or as described in U.S. Pub. No. 20120178702, incorporated herein by reference. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is incorporated herein by reference. In some embodiments, the immunogenic composition (e.g., vaccine composition) is formulated in a lipid nanoparticle that includes a non-cationic lipid such as, but not limited to, cholesterol or dioleoyl phosphatidylethanolamine (DOPE).

In some embodiments, a vaccine formulation described herein is a nanoparticle that comprises at least one lipid (termed a “lipid nanoparticle” or “LNP”). The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, incorporated herein by reference. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, the immunogenic composition (e.g., vaccine composition) described herein may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles may have a diameter from about 10 to 500 nm. In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the immunogenic composition (e.g., vaccine composition) is formulated in a liposome. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are incorporated herein by reference.

In some embodiments, the immunogenic composition (e.g., vaccine composition) described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; incorporated herein by reference) and liposomes which may deliver small molecule drugs such as, but not limited to, DOXIL® from Janssen Biotech, Inc. (Horsham, Pa.).

In some embodiments, the antigen and/or the adjuvantation system may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are incorporated herein by reference.

The antigen, the adjuvantation system, and/or optionally the second adjuvant may be formulated using any of the methods described herein or known in the art separately or together. For example, the antigen and the adjuvantation system may be formulated in one lipid nanoparticle or two separately lipid nanoparticles. In some embodiments, the antigen, the adjuvantation system are formulated in the same aqueous solution or two separate aqueous solutions.

Other aspects of the present disclosure provide methods of inducing an immune response to Beta coronavirus (e.g., MERS-CoV, SARS-CoV-1, or SARS-CoV-2) or a Beta coronavirus (e.g., MERS-CoV, SARS-CoV-1, or SARS-CoV-2) antigen in a subject in need thereof, the method comprising administering to the subject an effective amount of a Beta coronavirus (e.g., MERS-CoV, SARS-CoV-1, or SARS-CoV-2) antigen and an effective amount of an adjuvantation system comprising a PRR agonist (e.g., CpG-ODN, cGAMP). In some embodiments, the adjuvantation system further comprises alum. In some embodiments, the PRR agonist (e.g., CpG-ODN, cGAMP) is adsorbed into the alum.

In some embodiments, the adjuvantation system (e.g., comprising PRR agonist such as CpG-ODN or cGAMP alone or PRR agonist and alum) is administered separately from the Beta coronavirus antigen. In some embodiments, the adjuvantation system (e.g., comprising PRR agonist such as CpG-ODN or cGAMP alone or PRR agonist and alum) is administered prior to administering the Beta coronavirus antigen. In some embodiments, the adjuvantation system (e.g., comprising PRR agonist such as CpG-ODN or cGAMP alone or PRR agonist and alum) is administered after administering the Beta coronavirus antigen. In some embodiments, the adjuvantation system (e.g., comprising PRR agonist such as CpG-ODN or cGAMP alone or PRR agonist and alum) and the Beta coronavirus antigen are administered simultaneously. In some embodiments, the adjuvantation system (e.g., comprising PRR agonist such as CpG-ODN or cGAMP alone or PRR agonist and alum) and the Beta coronavirus antigen are administered as an admixture.

A “subject” to which administration is contemplated refers to a human (i.e., male or female of any age group, e.g., pediatric subject (e.g., infant, child, or adolescent) or adult subject (e.g., young adult, middle-aged adult, or senior (i.e. elderly) adult)) or non-human animal. In some embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In some embodiments, the non-human animal is a fish, reptile, or amphibian. The non-human animal may be a male or female at any stage of development. The non-human animal may be a transgenic animal or genetically engineered animal. A “subject in need thereof” refers to a subject (e.g., a human subject or a non-human mammal) in need of treatment of infection by a Beta coronavirus (e.g., a subject having MERS, SARS or COVID19) or in need of reducing the risk of developing an infection by Beta coronavirus (e.g., MERS-CoV, SARS-CoV-1, or SARS-CoV-2). In some embodiments, administering the Beta coronavirus (e.g., MERS-CoV, SARS-CoV-1, or SARS-CoV-2) antigen and the adjuvantation system described herein (e.g., comprising PRR agonist such as CpG-ODN or cGAMP alone or PRR agonist and alum) to a subject having Beta coronavirus (e.g., MERS-CoV, SARS-CoV-1, or SARS-CoV-2) infection treats (therapeutic use) the disease (MERS, SARS or COVID19). In some embodiments, administering the antigen and the adjuvantation system described herein (e.g., comprising PRR agonist such as CpG-ODN or cGAMP alone or PRR agonist and alum) to a subject at risk of developing an infection by a Beta coronavirus (e.g., MERS-CoV, SARS-CoV-1, or SARS-CoV-2) reduces the likelihood (e.g., by 20%, 30%, 40%, 50%, 60%, 70%, 80%, 90%, 95%, 99% or more) of the subject developing the infection (prophylactic use).

In some embodiments, the subject is a human subject, e.g., a human neonate, infant, child, adult, or elderly. In particular, the present disclosure demonstrates the immune enhancing effects of the adjuvantation system described herein (e.g., PRR agonist alone, or PRR agonist formulated with alum) in newborn human subjects. In some embodiments, the PRR agonist used in the adjuvantation system for enhancing an immune response to Beta coronavirus (e.g., MERS-CoV, SARS-CoV-1, or SARS-CoV-2) in a newborn human subject is Poly I:C, PHAD, CpG-ODN, or cGAMP (e.g., alone or formulated with alum). A “newborn” refers to a subject that is still in its infancy stage. For different species the infancy stage may be of different length. In some embodiments, the newborn subject is a human newborn. A human newborn refers to a human that is no more than one year of age (e.g., a human subject that is 1 hour, 12 hours, 1 day, 1 week, 1 month, 2 months, 3 months, 4 months, 5 months, 6 months, 7 months, 8 months, 9 months, 10 months, 11 months, or 12 months of age).

In some embodiments, the human newborn is a neonate that is less than 28 days of age at the time the vaccine described herein is administered. In some embodiments, the human neonate is 0-28 days, 0-27 days, 0-26 days, 0-25 days, 0-24 days, 0-23 days, 0-22 days, 0-21 days, 0-20 days, 0-19 days, 0-18 days, 0-17 days, 0-16 days, 0-15 days, 0-14 days, 0-13 days, 0-12 days, 0-11 days, 0-10 days, 0-9 days, 0-8 days, 0-7 days, 0-6 days, 0-5 days, 0-4 days, 0-3 days, 0-2 days, 0-1 days, 0-12 hours, 0-6 hours, 0-2 hours, 0-1 hour, 1-28 days, 1-27 days, 1-26 days, 1-25 days, 1-24 days, 1-23 days, 1-22 days, 1-21 days, 1-20 days, 1-19 days, 1-18 days, 1-17 days, 1-16 days, 1-15 days, 1-14 days, 1-13 days, 1-12 days, 1-11 days, 1-10 days, 1-9 days, 1-8 days, 1-7 days, 1-6 days, 1-5 days, 1-4 days, 1-3 days, 1-2 days, 2-28 days, 2-27 days, 2-26 days, 2-25 days, 2-24 days, 2-23 days, 2-22 days, 2-21 days, 2-20 days, 2-19 days, 2-18 days, 2-17 days, 2-16 days, 2-15 days, 2-14 days, 2-13 days, 2-12 days, 2-11 days, 2-10 days, 2-9 days, 2-8 days, 2-7 days, 2-6 days, 2-5 days, 2-4 days, 2-3 days, 3-28 days, 3-27 days, 3-26 days, 3-25 days, 3-24 days, 3-23 days, 3-22 days, 3-21 days, 3-20 days, 3-19 days, 3-18 days, 3-17 days, 3-16 days, 3-15 days, 3-14 days, 3-13 days, 3-12 days, 3-11 days, 3-10 days, 3-9 days, 3-8 days, 3-7 days, 3-6 days, 3-5 days, 3-4 days, 4-28 days, 4-27 days, 4-26 days, 4-25 days, 4-24 days, 4-23 days, 4-22 days, 4-21 days, 4-20 days, 4-19 days, 4-18 days, 4-17 days, 4-16 days, 4-15 days, 4-14 days, 4-13 days, 4-12 days, 4-11 days, 4-10 days, 4-9 days, 4-8 days, 4-7 days, 4-6 days, 4-5 days, 5-28 days, 5-27 days, 5-26 days, 5-25 days, 5-24 days, 5-23 days, 5-22 days, 5-21 days, 5-20 days, 5-19 days, 5-18 days, 5-17 days, 5-16 days, 5-15 days, 5-14 days, 5-13 days, 5-12 days, 5-11 days, 5-10 days, 5-9 days, 5-8 days, 5-7 days, 5-6 days, 6-28 days, 6-27 days, 6-26 days, 6-25 days, 6-24 days, 6-23 days, 6-22 days, 6-21 days, 6-20 days, 6-19 days, 6-18 days, 6-17 days, 6-16 days, 6-15 days, 6-14 days, 6-13 days, 6-12 days, 6-11 days, 6-10 days, 6-9 days, 6-8 days, 6-7 days, 7-28 days, 7-27 days, 7-26 days, 7-25 days, 7-24 days, 7-23 days, 7-22 days, 7-21 days, 7-20 days, 7-19 days, 7-18 days, 7-17 days, 7-16 days, 7-15 days, 7-14 days, 7-13 days, 7-12 days, 7-11 days, 7-10 days, 7-9 days, 7-8 days, 9-28 days, 9-27 days, 9-26 days, 9-25 days, 9-24 days, 9-23 days, 9-22 days, 9-21 days, 9-20 days, 9-19 days, 9-18 days, 9-17 days, 9-16 days, 9-15 days, 9-14 days, 9-13 days, 9-12 days, 9-11 days, 9-10 days, 10-28 days, 10-27 days, 10-26 days, 10-25 days, 10-24 days, 10-23 days, 10-22 days, 10-21 days, 10-20 days, 10-19 days, 10-18 days, 10-17 days, 10-16 days, 10-15 days, 10-14 days, 10-13 days, 10-12 days, 10-11 days, 11-28 days, 11-27 days, 11-26 days, 11-25 days, 11-24 days, 11-23 days, 11-22 days, 11-21 days, 11-20 days, 11-19 days, 11-18 days, 11-17 days, 11-16 days, 11-15 days, 11-14 days, 11-13 days, 11-12 days, 12-28 days, 12-27 days, 12-26 days, 12-25 days, 12-24 days, 12-23 days, 12-22 days, 12-21 days, 12-20 days, 12-19 days, 12-18 days, 12-17 days, 12-16 days, 12-15 days, 12-14 days, 12-13 days, 13-28 days, 13-27 days, 13-26 days, 13-25 days, 13-24 days, 13-23 days, 13-22 days, 13-21 days, 13-20 days, 13-19 days, 13-18 days, 13-17 days, 13-16 days, 13-15 days, 13-14 days, 14-28 days, 14-27 days, 14-26 days, 14-25 days, 14-24 days, 14-23 days, 14-22 days, 14-21 days, 14-20 days, 14-19 days, 14-18 days, 14-17 days, 14-16 days, 14-15 days, 15-28 days, 15-27 days, 15-26 days, 15-25 days, 15-24 days, 15-23 days, 15-22 days, 15-21 days, 15-20 days, 15-19 days, 15-18 days, 15-17 days, 15-16 days, 16-28 days, 16-27 days, 16-26 days, 16-25 days, 16-24 days, 16-23 days, 16-22 days, 16-21 days, 16-20 days, 16-19 days, 16-18 days, 16-17 days, 17-28 days, 17-27 days, 17-26 days, 17-25 days, 17-24 days, 17-23 days, 17-22 days, 17-21 days, 17-20 days, 17-19 days, 17-18 days, 18-28 days, 18-27 days, 18-26 days, 18-25 days, 18-24 days, 18-23 days, 18-22 days, 18-21 days, 18-20 days, 18-19 days, 19-28 days, 19-27 days, 19-26 days, 19-25 days, 19-24 days, 19-23 days, 19-22 days, 19-21 days, 19-20 days, 20-28 days, 20-27 days, 20-26 days, 20-25 days, 20-24 days, 20-23 days, 20-22 days, 20-21 days, 21-28 days, 21-27 days, 21-26 days, 21-25 days, 21-24 days, 21-23 days, 21-22 days, 22-28 days, 22-27 days, 22-26 days, 22-25 days, 22-24 days, 22-23 days, 23-28 days, 23-27 days, 23-26 days, 23-25 days, 23-24 days, 24-28 days, 24-27 days, 24-26 days, 24-25 days, 25-28 days, 25-27 days, 25-26 days, 26-28 days, 26-27 days, or 27-28 days of age at the time of administration of the immunogenic composition (e.g., vaccine composition) described herein. In some embodiments, the human neonate is 1, 2, 3, 4, 5, 6, 7, 8, 9, 10, 11, 12, 13, 14, 15, 16, 17, 18, 19, 20, 21, 22, 23, 24, 25, 26, 27, or 28 days of age at the time of administration of the immunogenic composition (e.g., vaccine composition) described herein.

In some embodiments, the human infant is less than 28 days of age at the time of administration (vaccination). In some embodiments, the human infant is less than 4 days of age at the time of administration (vaccination). In some embodiments, the human infant is less than 2 days of age at the time of administration (vaccination). In some embodiments, the human infant is less than 24 hours of age at the time of administration (vaccination). In some embodiments, the administration (vaccination) occurs at birth. In some embodiments, a human neonate (less than 28 days of age) receives 1 or 2 doses of the vaccine described herein. In some embodiments, the human neonate receives one dose before 28-days of age (e.g., 2, 3, 4, 5, 6, 7, 8, 9, 10, 11, 12, 13, 14, 15, 16, 17, 18, 19, 20 days of age) and a second dose before or at 28-days of age. In some embodiments, the human subject receives one dose at 2 months, 4 months, or 6 months of age, and a second dose after the first dose at 2 months, 4 months, or 6 months of age. In some embodiments, a human subject receives a second dose before or equal to 6-months of age (e.g., 1, 2, 3, 4, 5, 6 months of age). In some embodiments, the administration occurs when the human infant is 2 months, 4 months, and 6 months of age. In some embodiments, a human subject receives a second dose after 6-months of age (e.g., 1 year, 2 years, 3 years of age).

In some embodiments, immunization of older human subjects that are more than 28-days old (e.g., 1 month, 2 months, 3 months, 4 months, 5 months, 6 months, 7 months, 8 months, 9 months, 10 months, 11 months, 12 months, 2 years, 3 years, 4 years, 5 years, 10 years, 11 years, 12 years, 13 years, 14 years, 15 years, 16 years, 17 years old) is contemplated. In some embodiments, the human subject is an adult (e.g., more than 18 years old). In some embodiments, the human subject is an elderly (e.g., more than 60 years old). In some embodiments, the human subject is more than 65-years of age. In some embodiments, the human subject receives one or two doses of the vaccine described herein after 65-years of age.

In some embodiments, the human subject is born prematurely or has low birth weight. “Born prematurely” means the human subject is born before 40-weeks of term. In some embodiments, the human subject is born before 37-weeks of term. In some embodiments, the human subject is born before 32 weeks of term. In some embodiments, the human subject is born before 24 weeks of term. In some embodiments, the human subject is born before 40 weeks, 39 weeks, 38 weeks, 37 weeks, 36 weeks, 35 weeks, 34 weeks, 33 weeks, 32 weeks, 31 weeks, 30 weeks, 29 weeks, 28 weeks, 27 weeks, 26 weeks, 25 weeks, or 24 weeks of term. In some embodiments, the human subject is born with low birth weight (e.g., at least 20% lower than a normal birth weight).

In some embodiments, the human subject is more than 28 days of age (e.g., 1 month, 2 months, 3 months, 4 months, 5 months, 6 months, 7 months, 8 months, 9 months, 10 months, 11 months, 12 months, 2 years, 3 years, 4 years, 5 years, 10 years, 11 years, 12 years, 13 years, 14 years, 15 years, 16 years, 17 years of age). In some embodiments, the human subject is an adult (e.g., more than 18 years of age). In some embodiments, the human subject is an elderly subject (e.g., more than 60 years of age). In some embodiments, the human subject is 60 years, 65 years, 70 years, 75 years, 80 years, 85 years, 90 years, 95 years, 100 years, or more than 100 years of age.

In some embodiments, a human subject receives 1, 2, or more than 2 doses of the vaccine described herein. In some embodiments, a human neonate receives one dose before 28 days of age (e.g., 2, 3, 4, 5, 6, 7, 8, 9, 10, 11, 12, 13, 14, 15, 16, 17, 18, 19, 20 days of age) and a second dose before or after 28-days of age. In some embodiments, a human subject receives one dose before 60 years of age and a second dose before, at, or after 60 years of age (e.g., 60, 65, 70, 75, 80, 85, 90, 95, 100, or more than 100 years of age, or any age therebetween as if explicitly recited). In some embodiments, the human subject receives a second dose of the vaccine 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years or more after receiving the first dose.

In some embodiments, the human subject has an undeveloped (e.g., an infant or a neonate), weak (an elderly), or compromised immune system. Immunocompromised subjects include, without limitation, subjects with primary immunodeficiency or acquired immunodeficiency such as those suffering from sepsis, HIV infection, and cancers, including those undergoing chemotherapy and/or radiotherapy. In some embodiments, the human subject has an underlying condition that renders them more susceptible to Beta coronavirus (e.g., MERS-CoV, SARS-CoV-1, or SARS-CoV-2) infection. In some embodiments, the human subject is immune-compromised, has chronic lung disease, asthma, cardiovascular disease, cancer, obesity, diabetes, chronic kidney disease, and/or liver disease.

In some embodiments, the subject is a companion animal (a pet). The use of the immunogenic compositions (e.g., vaccine compositions) described herein in veterinary vaccine is also within the scope of the present disclosure. “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include: rodents (e.g., ferrets, pigs, rats, mice, guinea pigs, and hamsters), rabbits, or non-human primates.

Once administered, the immunogenic composition (e.g., vaccine composition) described herein elicits an immune response in the subject. In some embodiments, the immune response is an innate immune response. In some embodiments, the immune response is an adaptive immune response specific to the antigen in the composition or vaccine. In some embodiments, the immunogenic composition (e.g., vaccine composition) described herein activates B cell immunity. In some embodiments, the immunogenic composition (e.g., vaccine composition) elicits production of antibodies against the antigen. In some embodiments, the immunogenic composition (e.g., vaccine composition) activates cytotoxic T cells specific to the antigen.

In some embodiments, the adjuvantation system described herein (e.g., PRR agonist alone, or PRR agonist formulated with alum), whether administered alone or in an admixture with an Beta coronavirus antigen, enhance the innate immune response, compared to without the adjuvantation system or when the Beta coronavirus antigen is administered alone. In some embodiments, the adjuvantation system described herein (e.g., PRR agonist alone, or PRR agonist formulated with alum) activates newborn or elderly peripheral blood mononuclear cells (PBMCs). In some embodiments, the number of PBMCs that are activated is increased by at least 20% in the presence of the adjuvantation system described herein (e.g., PRR agonist alone, or PRR agonist formulated with alum), compared to without the adjuvantation system or when the Beta coronavirus antigen is administered alone. For example, the number of PBMCs that are activated may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the adjuvantation system, compared to without the adjuvantation system or when the Beta coronavirus antigen is administered alone. In some embodiments, the number of PBMCs that are activated is increased by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the Beta coronavirus antigen is administered alone.

In some embodiments, the adjuvantation system described herein (e.g., PRR agonist alone, or PRR agonist formulated with alum) enhances the production of a proinflammatory cytokine (e.g., IL-2, IL-6, IL-10, TNF, IFNα, IFNγ, CCL3, CXCL8, GM-CSF) in the subject. In some embodiments, the level of proinflammatory cytokines (e.g., IL-2, IL-6, IL-10, TNF, IFNα, IFNγ, CCL3, CXCL8, GM-CSF) is increased by at least 20% in the presence of the adjuvantation system, compared to without the adjuvantation system or when the Beta coronavirus antigen is administered alone. For example, the level of proinflammatory cytokines (e.g., IL-2, IL-6, IL-10, TNF, IFNα, IFNγ, CCL3, CXCL8, GM-CSF) may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the adjuvantation system, compared to without the adjuvantation system or when the Beta coronavirus antigen is administered alone. In some embodiments, the level of proinflammatory cytokines (e.g., IL-2, IL-6, IL-10, TNF, IFNα, IFNγ, CCL3, CXCL8, GM-CSF) is increased by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the Beta coronavirus antigen is administered alone.

In some embodiments, the adjuvantation system enhances innate immune memory (also referred to as trained immunity). “Innate immune memory” confers heterologous immunity that provides broad protection against a range of pathogens. In some embodiments, the innate immune memory is increased by at least 20% in the presence of the adjuvantation system, compared to without the adjuvantation system or when the Beta coronavirus antigen is administered alone. For example, the innate immune memory may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the adjuvantation system, compared to without the adjuvantation system or when the Beta coronavirus antigen is administered alone. In some embodiments, the innate immune memory is increased by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the Beta coronavirus antigen is administered alone.

In some embodiments, the adjuvantation system, when administered as an admixture with a Beta coronavirus antigen, enhances the anti-specific immune response against the Beta coronavirus (e.g., MERS-CoV, SARS-CoV-1, or SARS-CoV-2) antigen or against the Beta coronavirus (e.g., MERS-CoV, SARS-CoV-1, or SARS-CoV-2), compared to without the adjuvantation system or when the Beta coronavirus antigen is administered alone. In some embodiments, the adjuvantation system enhances the production of antigen-specific antibody titer (e.g., by at least 20%) in the subject, compared to without the adjuvantation system or when the Beta coronavirus antigen is administered alone. For example, the adjuvantation system may enhance the production of antigen-specific antibody titer by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. in the subject, compared to without the adjuvantation system or when the Beta coronavirus antigen is administered alone. In some embodiments, the adjuvantation system enhances the production of antigen-specific antibody titer by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the Beta coronavirus antigen is administered alone. One skilled in the art is familiar with how to evaluate the level of an antibody titer, e.g., by ELISA. In some embodiments, the antigen-specific antibody for which production is enhanced is an immunoglobulin A (IgA), immunoglobulin D (IgG), immunoglobulin E (IgE), immunoglobulin G (IgG), or immunoglobulin M (IgM). In some embodiments, the antigen-specific antibody is an IgG. In some embodiments, the antigen-specific antibody is a subclass 1 IgG (IgG1), subclass 2 IgG (IgG2), subclass 3 IgG (IgG3), or subclass 4 IgG (IgG4).

In some embodiments, the adjuvantation system enhances the production of antigen-specific antibodies that neutralize (i.e., render non-infectious) Beta coronavirus particles. In some embodiments, the adjuvantation system enhances the neutralizing antibody titer by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the Beta coronavirus antigen is administered alone. In some embodiments, the adjuvantation system enhances the antigen-specific antibody titer capable of neutralizing a particular Beta coronavirus variant, compared to without the adjuvantation system or when the Beta coronavirus antigen is administered alone. In some embodiments, the variant is wild-type MERS-CoV, SARS-CoV-1, or SARS-CoV-2. In some embodiments, the variant is a Beta coronavirus variant that is not a wild-type variant. In some embodiments, the variant is a variant of SARS-CoV-2 that is not wild-type SARS-CoV-2, such as, but not limited to, B.1.1.7, B.1.351, P.1, B.1.427, B.1.429, B.1.526, B.1.526.1, B.1.525, P.2, B.1.617, B.1.617.1, B.1.617.2, or B.1.617.3 SARS-CoV-2.

In some embodiments, the adjuvantation system polarizes the innate and adaptive immune response by shaping the pattern of cytokine and/or chemokine responses toward T helper 1 (Th1) immunity, important for host defense against intracellular pathogens. In some embodiments, the adjuvantation system polarizes the innate immune response toward T follicular helper (Tfh) cell immunity.

In some embodiments, the adjuvantation system enhances the inhibition of interaction between angiotensin-converting enzyme 2 (ACE2) expressed by a subject and Beta coronavirus spike protein, compared to without the adjuvantation system or when the Beta coronavirus antigen is administered alone. For example, the adjuvantation system may enhance the inhibition of interaction between ACE2 expressed by a subject and Beta coronavirus spike protein by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the Beta coronavirus antigen is administered alone. In the presence of the adjuvantation system, interaction between ACE2 expressed by a subject and Beta coronavirus spike protein may be reduced by 10%, 20%, 30%, 40%, 50%, 60%, 70%, 80%, 90%, 95%, 99%, or more than 99%, compared to without the adjuvantation system or when the Beta coronavirus antigen is administered alone.

In some embodiments, the adjuvantation system prolongs the effect of a vaccine (e.g., by at least 20%) in the subject, compared to without the adjuvantation system or when the Beta coronavirus antigen is administered alone. For example, the adjuvantation system may prolong the effect of a vaccine by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. in the subject, compared to without the adjuvantation system or when the Beta coronavirus antigen is administered alone. In some embodiments, the adjuvantation system prolongs the effect of a vaccine by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the Beta coronavirus antigen is administered alone.

In some embodiments, the adjuvantation system increases rate of (accelerates) an immune response, compared to without the adjuvantation system or when the Beta coronavirus antigen is administered alone. For example, the adjuvantation system may increase the rate of an immune response by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. in the subject, compared to without the adjuvantation system or when the Beta coronavirus antigen is administered alone. In some embodiments, the adjuvantation system increases the rate of an immune response by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the Beta coronavirus antigen is administered alone. “Increase the rate of immune response” mean it takes less time for the immune system of a subject to react to an invading Beta coronavirus (e.g., MERS-CoV, SARS-CoV-1, or SARS-CoV-2).

In some embodiments, the antigen produces a same level of immune response against the Beta coronavirus (e.g., MERS-CoV, SARS-CoV-1, or SARS-CoV-2) antigen at a lower dose in the presence of the adjuvantation system, compared to without the adjuvantation system or when the Beta coronavirus antigen is administered alone. In some embodiments, the amount of Beta coronavirus antigen needed to produce the same level of immune response is reduced by at least 20% in the presence of the adjuvantation system, compared to without the adjuvantation system or when the Beta coronavirus antigen is administered alone. For example, the amount of antigen needed to produce the same level of immune response may be reduced by at least 20%, at least 30%, at least 40%, at least 50%, at least 60%, at least 70%, at least 80%, at least 90%, at least 95%, at least 99% or more, in the presence of the adjuvantation system, compared to without the adjuvantation system or when the Beta coronavirus antigen is administered alone. In some embodiments, the amount of antigen needed to produce the same level of immune response is reduced by at 20%, 30%, 40%, 50%, 60%, 70%, 80%, 90%, 95%, 99% or more, in the presence of the adjuvantation system, compared to without the adjuvantation system or when the Beta coronavirus antigen is administered alone.

The prophylactic or therapeutic use of the adjuvantation system, or the immunogenic composition (e.g., vaccine composition) described herein is also within the scope of the present disclosure. In some embodiments, the composition or immunogenic composition (e.g., vaccine composition) described herein are used in methods of vaccinating a subject by prophylactically administering to the subject an effective amount of the composition or immunogenic composition (e.g., vaccine composition) described herein. “Vaccinating a subject” refer to a process of administering an immunogen, typically an antigen formulated into a vaccine, to the subject in an amount effective to increase or activate an immune response against the Beta coronavirus antigen (e.g., MERS-COV, SARS-COV-1, SARS-COV-2) and, thus, against Beta coronavirus (e.g., MERS-COV, SARS-COV-1, SARS-COV-2). In some embodiments, the terms do not require the creation of complete immunity against SARS-CoV. In some embodiments, the terms encompass a clinically favorable enhancement of an immune response toward the Beta coronavirus antigen or pathogen. Methods for immunization, including formulation of a immunogenic composition (e.g., vaccine composition) and selection of doses, routes of administration and the schedule of administration (e.g. primary dose and one or more booster doses), are well known in the art. In some embodiments, vaccinating a subject reduces the risk of developing Beta coronavirus (e.g., MERS-CoV, SARS-CoV-1, or SARS-CoV-2) infection and the resulting disease (e.g., MERS, SARS and/or COVID19)

In some embodiments, the immunogenic compositions (e.g., vaccine composition) described herein are formulated for administration to a subject. In some embodiments, the composition or immunogenic composition (e.g., vaccine composition) further comprises a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the composition or immunogenic composition (e.g., vaccine composition) described herein also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The immunogenic composition (e.g., vaccine composition) described herein may conveniently be presented in unit dosage form and may be prepared by any of the methods well-known in the art of pharmacy. The term “unit dose” when used in reference to a composition or immunogenic composition (e.g., vaccine composition) described herein of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the composition or immunogenic compositions (e.g., vaccine composition) described herein may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For topical administration, the composition or immunogenic composition (e.g., vaccine composition) described herein can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, an elixir, or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the immunogenic composition (e.g., vaccine composition) described herein used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The cyclic Psap peptide and/or the composition or immunogenic composition (e.g., vaccine composition) described herein ordinarily will be stored in lyophilized form or as an aqueous solution if it is highly stable to thermal and oxidative denaturation. The pH of the preparations typically will be about from 6 to 8, although higher or lower pH values can also be appropriate in certain instances. The chimeric constructs of the present disclosure can be used as vaccines by conjugating to soluble immunogenic carrier molecules. Suitable carrier molecules include protein, including keyhole limpet hemocyanin, which is a preferred carrier protein. The chimeric construct can be conjugated to the carrier molecule using standard methods. (Hancock et al., “Synthesis of Peptides for Use as Immunogens,” in Methods in Molecular Biology: Immunochemical Protocols, Manson (ed.), pages 23-32 (Humana Press 1992)).

In some embodiments, the present disclosure contemplates an immunogenic composition (e.g., vaccine composition) comprising a pharmaceutically acceptable injectable vehicle. The vaccines of the present disclosure may be administered in conventional vehicles with or without other standard carriers, in the form of injectable solutions or suspensions. The added carriers might be selected from agents that elevate total immune response in the course of the immunization procedure.

Liposomes have been suggested as suitable carriers. The insoluble salts of aluminum, that is aluminum phosphate or aluminum hydroxide, have been utilized as carriers in routine clinical applications in humans. Polynucleotides and polyelectrolytes and water-soluble carriers such as muramyl dipeptides have been used.

Preparation of injectable vaccines of the present disclosure, includes mixing the immunogenic composition (e.g., vaccine composition) with muramyl dipeptides or other carriers. The resultant mixture may be emulsified in a mannide monooleate/squalene or squalane vehicle. Four parts by volume of squalene and/or squalane are used per part by volume of mannide monooleate. Methods of formulating immunogenic composition (e.g., vaccine composition)s are well-known to those of ordinary skill in the art. (Rola, Immunizing Agents and Diagnostic Skin Antigens. In: Remington's Pharmaceutical Sciences, 18th Edition, Gennaro (ed.), (Mack Publishing Company 1990) pages 1389-1404).

Additional pharmaceutical carriers may be employed to control the duration of action of a vaccine in a therapeutic application. Control release preparations can be prepared through the use of polymers to complex or adsorb chimeric construct. For example, biocompatible polymers include matrices of poly(ethylene-co-vinyl acetate) and matrices of a polyanhydride copolymer of a stearic acid dimer and sebacic acid. (Sherwood et al. (1992) Bio/Technology 10: 1446). The rate of release of the chimeric construct from such a matrix depends upon the molecular weight of the construct, the amount of the construct within the matrix, and the size of dispersed particles. (Saltzman et al. (1989) Biophys. J. 55: 163; Sherwood et al, supra.; Ansel et al. Pharmaceutical Dosage Forms and Drug Delivery Systems, 5th Edition (Lea & Febiger 1990); and Gennaro (ed.), Remington's Pharmaceutical Sciences, 18th Edition (Mack Publishing Company 1990)). The chimeric construct can also be conjugated to polyethylene glycol (PEG) to improve stability and extend bioavailability times (e.g., Katre et al.; U.S. Pat. No. 4,766,106).

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. Prophylactic treatment refers to the treatment of a subject who is not and was not with a disease but is at risk of developing the disease or who was with a disease, is not with the disease, but is at risk of regression of the disease. In some embodiments, the subject is at a higher risk of developing the disease or at a higher risk of regression of the disease than an average healthy member of a population.

An “effective amount” of a composition described herein refers to an amount sufficient to elicit the desired biological response. An effective amount of a composition described herein may vary depending on such factors as the desired biological endpoint, the pharmacokinetics of the compound, the condition being treated, the mode of administration, and the age and health of the subject. In some embodiments, an effective amount is a therapeutically effective amount. In some embodiments, an effective amount is a prophylactic treatment. In some embodiments, an effective amount is the amount of a compound described herein in a single dose. In some embodiments, an effective amount is the combined amounts of a compound described herein in multiple doses. When an effective amount of a composition is referred herein, it means the amount is prophylactically and/or therapeutically effective, depending on the subject and/or the disease to be treated. Determining the effective amount or dosage is within the abilities of one skilled in the art.

The terms “administer,” “administering,” or “administration” refers to implanting, absorbing, ingesting, injecting, inhaling, or otherwise introducing a compound described herein, or a composition thereof, in or on a subject. The composition of the immunogenic composition (e.g., vaccine composition) described herein may be administered systemically (e.g., via intravenous injection) or locally (e.g., via local injection). In some embodiments, the composition of the immunogenic composition (e.g., vaccine composition) described herein is administered orally, intravenously, topically, intranasally, or sublingually. Parenteral administration is also contemplated. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some embodiments, the composition is administered prophylactically.

In some embodiments, the composition or immunogenic composition (e.g., vaccine composition) is administered once or multiple times (e.g., 2, 3, 4, 5, or more times). For multiple administrations, the administrations may be done over a period of time (e.g., 6 months, a year, 2 years, 5 years, 10 years, or longer). In some embodiments, the composition or immunogenic composition (e.g., vaccine composition) is administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later).

### EXAMPLES

### Example 1—Adjuvantation of SARS-CoV-1 Antigen with a STING Agonist

Human immunity is crucial to both health and illness, playing key roles in multiple major diseases including infectious diseases, allergy and cancer. In this context there is growing interest in development of approaches to modulate the human immune system to prevent and/or treat illness. Infectious diseases are a leading cause of morbidity and mortality at the extremes of life. Immunization is a key strategy for preventing infectious diseases, but immunization of distinct vulnerable populations such as the young and elderly may result in sub-optimal responses, often requiring multiple booster doses and can be limited by waning immunity.

Adjuvantation is a key approach to enhance vaccine-induced immunity. Adjuvants can enhance, prolong, and modulate immune responses to vaccinal antigens to maximize protective immunity, and may potentially enable effective immunization in vulnerable populations (e.g., in the very young and the elderly or for diseases lacking effective vaccines). SARS-CoV-2, the causal agent of COVID-19, first emerged in late 2019 in China. As of the end of May 2020, it had infected almost 6,000,000 individuals and caused >350,000 deaths globally, especially in the elderly population. A year later, it has infected almost 160 million individuals and caused >3,300,000 deaths globally. Discovery, development and implementation of safe and effective vaccines will be key to addressing the SARS-coronavirus-2 (SARS-CoV-2) pandemic.

It is reported here that the adjuvantation system comprising a combination of the STING agonist 2′3′-cGAMP with alhydrogel (alum), enhances the anti-IgG antibody (Ab) response against SARS-CoV-1 receptor binding domain (RBD) of the spike glycoprotein (FIG. 1).

Efforts to develop an adjuvanted CoV vaccine with the spike protein receptor binding domain (S-RBD) of SARS-CoV-1 as vaccinal antigen as it was readily available and it is ˜75% identical at the amino acid level with the S-RBD of the current SARS-CoV-2 S-RBD. RBD is important to mediate SARS-CoV interaction with its receptor ACE2 and cellular entry, therefore playing a critical role in SARS-CoV infectivity and antibodies against RBD prevent CoV infectivity in vitro and in vivo. Even though antibodies against the spike glycoproteins of other CoVs can cross-react with SARS-CoV-2 spike glycoprotein, it still needs to be determined whether cross-reactivity will translate into clinical protection.

Furthermore, it has recently been suggested that antibodies against SARS-CoV-2 spike and RBD proteins are almost absent in SARS-CoV-2-uninfected individuals. Therefore, SARS-CoV-2 represents unique threat to human health that requires novel preventive and therapeutic approaches. The adjuvantation system described herein (e.g., 2′3′-cGAMP with alhydrogel (alum)) combined with SARS-CoV-2 RBD protein can be a highly immunogenic vaccine for the elderly by enhancing anti-SARS-CoV-2 RBD IgG production and promoting type 1 immunity.

### Example 2—Adjuvantation of SARS-CoV-2 Antigen with PRR Agonists

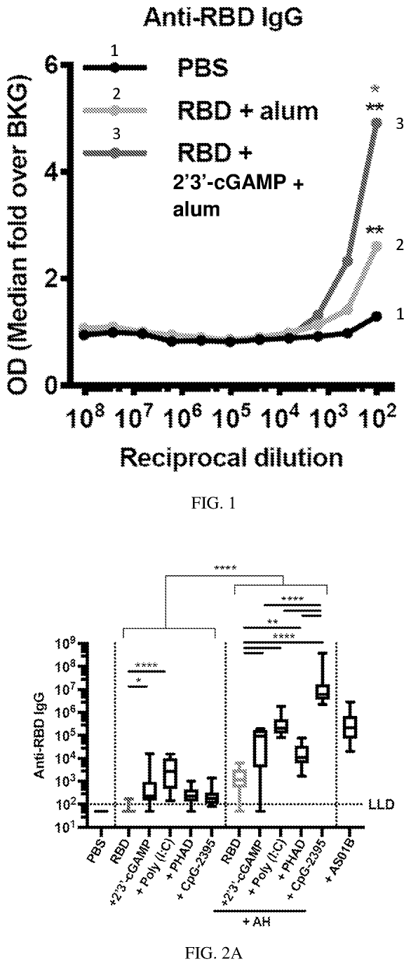

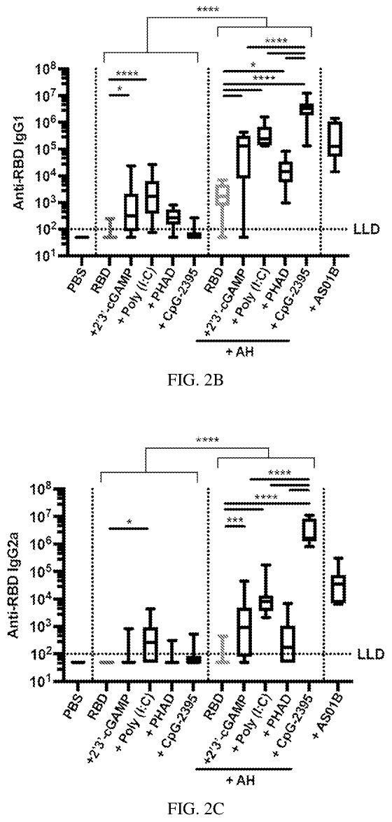

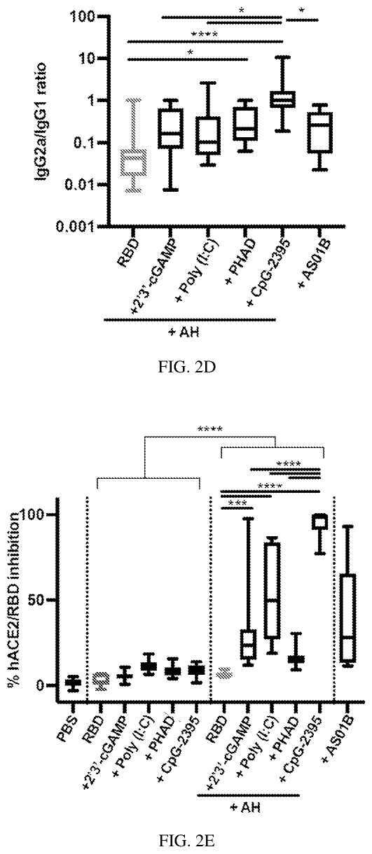

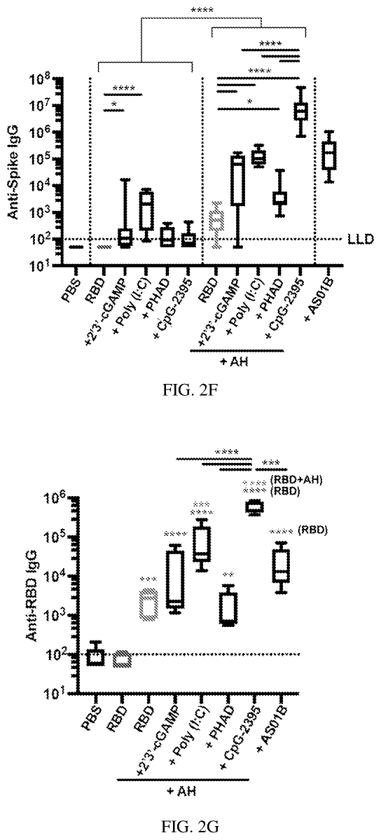

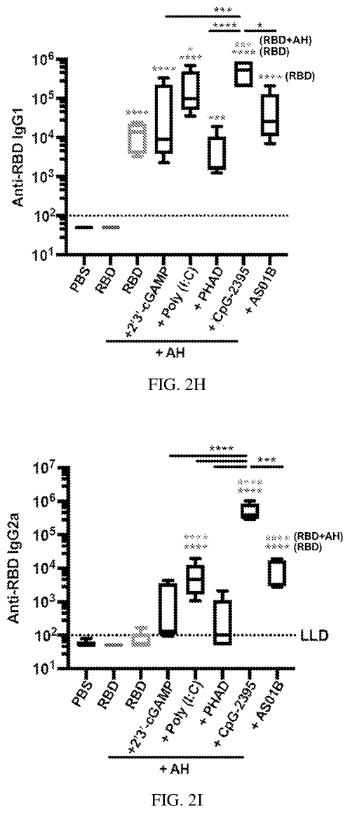

It was evaluated whether distinct aluminum hydroxide:pattern recognition receptor agonist (AH:PRR-A) formulations can overcome the low immunogenicity of monomeric RBD protein. To this end, a comprehensive comparison of PRR-As was performed, including 2′3′-cGAMP (a stimulator of IFN genes (STING) ligand), Poly (I:C) (a TLR3 agonist), PHAD (synthetic MPLA, a TLR4 agonist), and CpG-ODN 2395 (a TLR9 agonist). Each PRR-A was formulated with and without AH. AS01B (a liposome-based adjuvant containing MPLA and saponin QS-21) was also included as a clinical-grade benchmark adjuvant with potent immunostimulatory activity. The immunogenicity of vaccine formulations was first evaluated in young BALB/c mice (3-month-old). Mice were immunized intramuscularly twice with 10 μg of monomeric RBD protein formulated with or without adjuvant, in a two-dose prime-boost regimen (days 0 and 14). Two weeks following the boost immunization, humoral immune responses were evaluated. AH:PRR-A formulations enhanced anti-RBD Ab titers and inhibition of RBD binding to human ACE2 (hACE2) compared to their respective non-AH adjuvanted formulations (FIGS. 2A-2C). The Ab response elicited by AH without PRR-As was highly skewed to IgG1 (FIG. 2D), with minimal inhibition of hACE2/RBD binding (FIG. 2E). Among various AH:PRR-A formulations, AH:CpG demonstrated the highest induction of total IgG, IgG1, and IgG2a with a balanced IgG2a/IgG1 ratio (FIGS. 2A-2D). Furthermore, AH:CpG formulation significantly enhanced hACE2/RBD binding inhibition compared to all the other AH:PRR-A formulations (FIG. 2E). Antibodies induced by monomeric RBD immunization recognized the native trimeric spike protein as demonstrated by a binding ELISA with prefusion stabilized form of spike trimer (FIG. 2F). While immune responses to other adjuvanted formulations demonstrated waning immunity by Day 210, in contrast, AH:CpG formulations induced robust and durable anti-RBD titer and hACE2/RBD binding inhibition (FIG. 2G-2J).

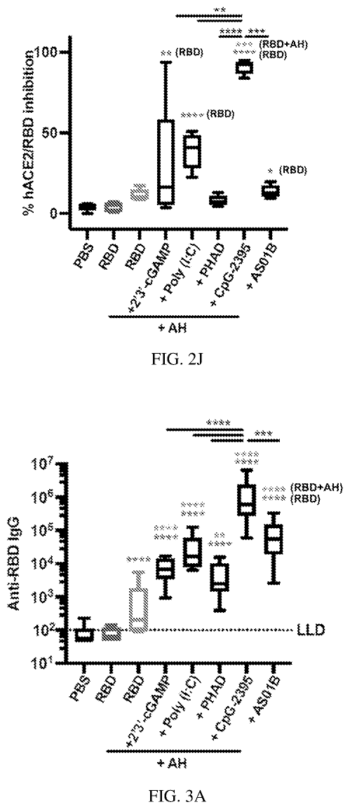

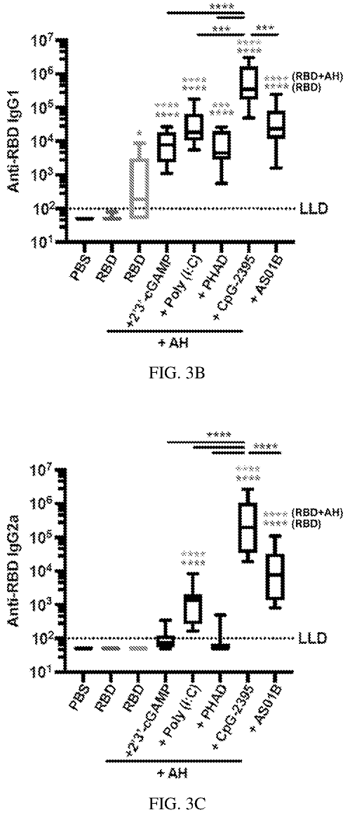

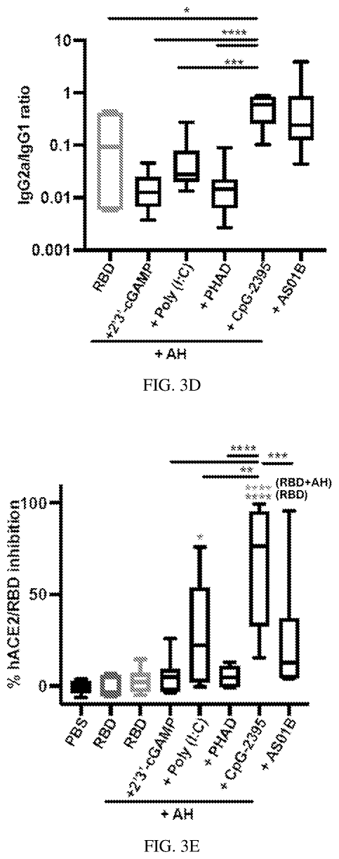

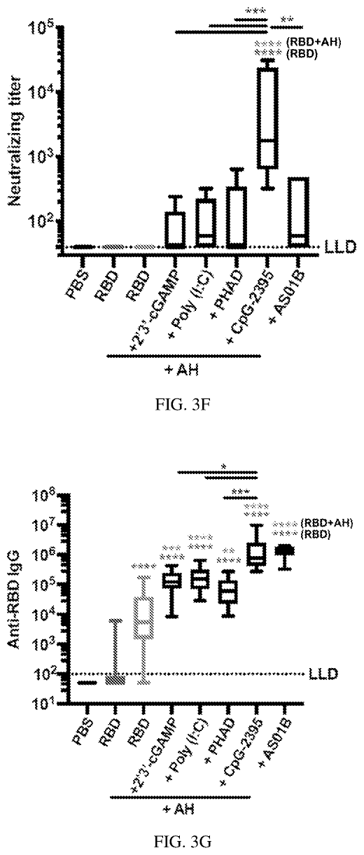

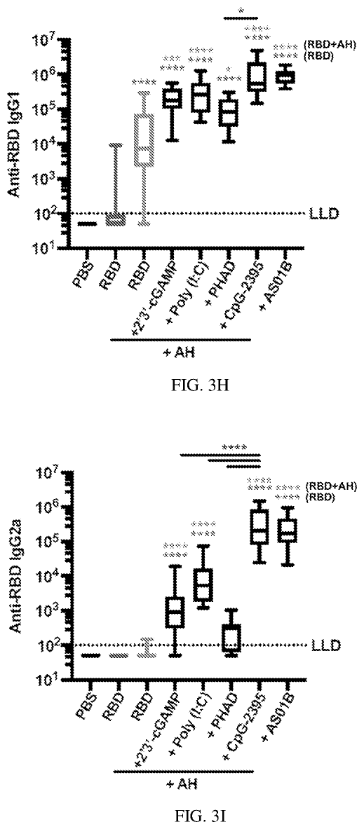

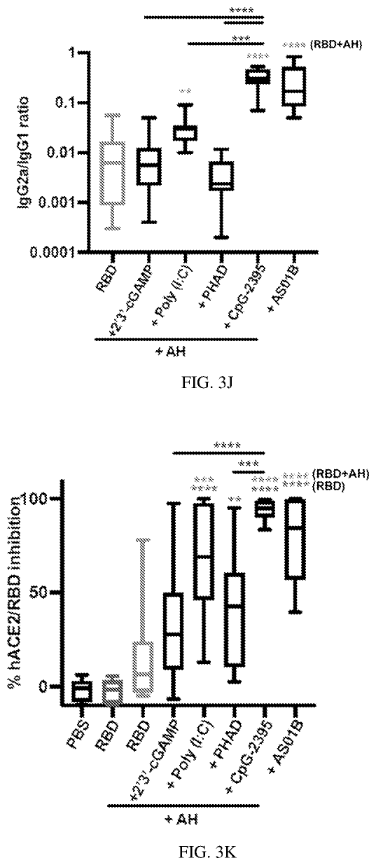

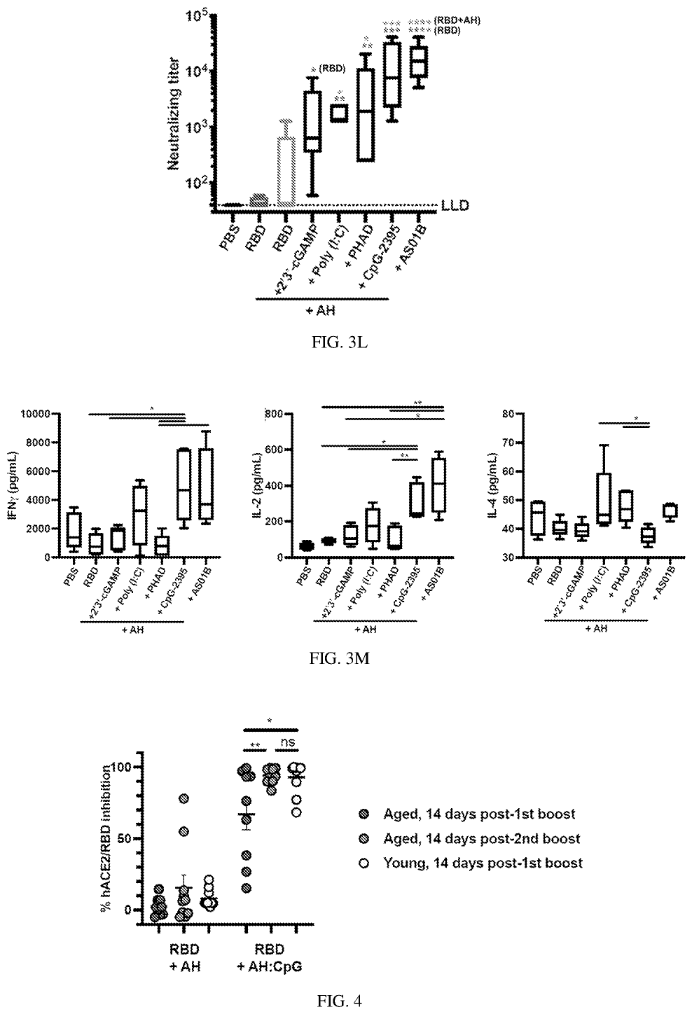

To assess the vaccine response in the context of aging, the immunogenicity of RBD vaccines adjuvanted with AH:PRR-A was further studied in aged mice (14-month-old). Similar to young mice, AH:CpG formulation elicited the highest humoral immune response after prime-boost immunization (FIGS. 3A-3F). Of note, the vaccine adjuvanted with AH:CpG elicited significantly higher hACE2/RBD inhibition and neutralizing titers over the vaccine adjuvanted with AS01B, which is known as a potent adjuvant in the human elderly population^{33,34} (FIGS. 3E-3F). However, antibody levels were generally lower in aged mice, and the magnitude of the immune response of aged mice receiving AH:CpG was significantly lower than the level of young mice, suggesting an impaired vaccine response due to immunosenescence in the elderly population (FIG. 4). To determine whether an additional dose can improve vaccine immunogenicity in aged mice, a second booster dose was administered two weeks after the last immunization. On Day 42 (two weeks after the second boost), enhancement in humoral responses was observed in AH:PRR-A formulations (FIGS. 3G-3L). Notably, significant enhancement of hACE2/RBD inhibition was observed in aged mice receiving AH:CpG formulation, which reached the level of young mice receiving AH:CpG with prime-boost regimen (FIG. 4). High serum concentrations of neutralizing Ab titers were observed in AH:CpG and AS01B adjuvanted groups after the second boost, but not in non-adjuvanted nor AH alone-adjuvanted RBD groups. Assessment of cytokine production by splenocytes isolated from immunized mice and restimulated in vitro with Spike peptides demonstrated high Th1 (IFNγ and IL-2) and low Th2 (IL-4) cytokine production in the AH:CpG and AS01B groups (FIG. 3M). These results demonstrate that AH:CpG-adjuvanted RBD vaccine is highly immunogenic in aged mice, and an additional booster dose can further enhance anti-RBD humoral responses to match those of younger subjects.

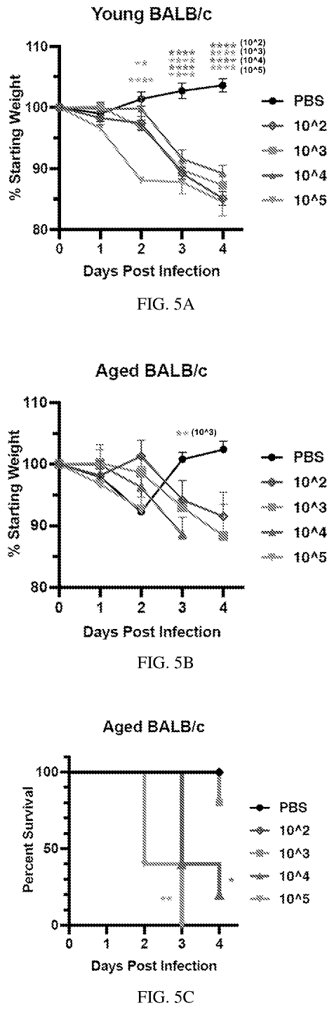

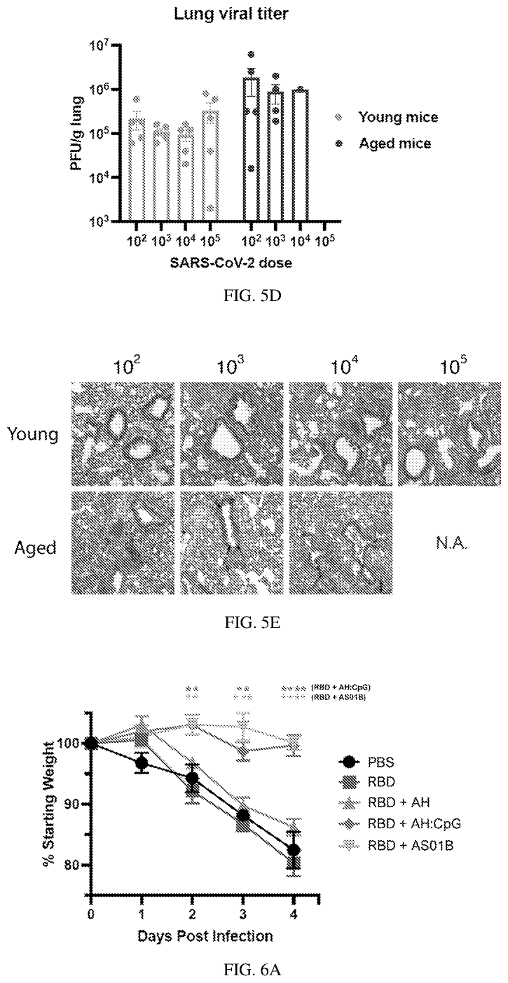

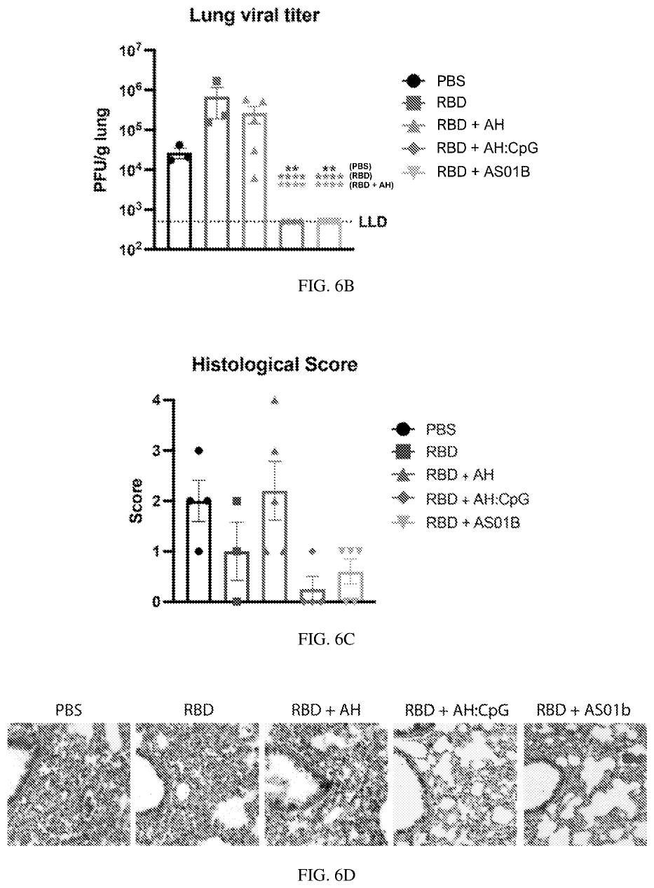

Neutralizing antibodies are key to protecting from SARS-CoV-2 infection. Since RBD formulated with AH:CpG elicited high titers of neutralizing Abs, protection of immunized mice in a challenge model was assessed. To this end, the mouse-adapted SARS-CoV-2 MA10 virus strain was employed³⁵. When tested in young (3-month-old) and aged (14-month-old) BALB/c mice, SARS-CoV-2 MA10 elicited dose-dependent weight loss (FIGS. 5A-5B). Notably, aged mice challenged with 10³ PFU or over exhibited dose-dependent mortality by 4 days post infection (dpi) (FIG. 5C). In comparison to aged mice, none of the young mice died by 4 dpi, including those received the highest viral dose. Next, immunized aged mice were challenged with SARS-CoV-2 MA10 six weeks after the second boost. Body weight changes were assessed daily up to 4 dpi, when the mice were sacrificed for viral load and histopathology analyses. Aged mice immunized with AH:CpG and AS01B adjuvanted vaccine did not show weight loss up to 4 dpi, whereas aged mice immunized with non-adjuvanted, or AH-adjuvanted RBD observed rapid and significant body weight loss of >10% through 4 dpi (FIG. 6A). Lung tissues were harvested and tested for SARS-CoV-2 viral loads. Complete sterilization of viral loads in lung tissues was observed in AH:CpG and AS01B adjuvanted groups, while viral loads were detectable in the vehicle, non-adjuvanted, or AH-adjuvanted groups (FIG. 6B). Histopathological analysis conducted in lung tissues further confirmed the reduced SARS-CoV-2 infection in aged animals vaccinated with AH:CpG and AS01B adjuvants (FIG. 6C-6D).

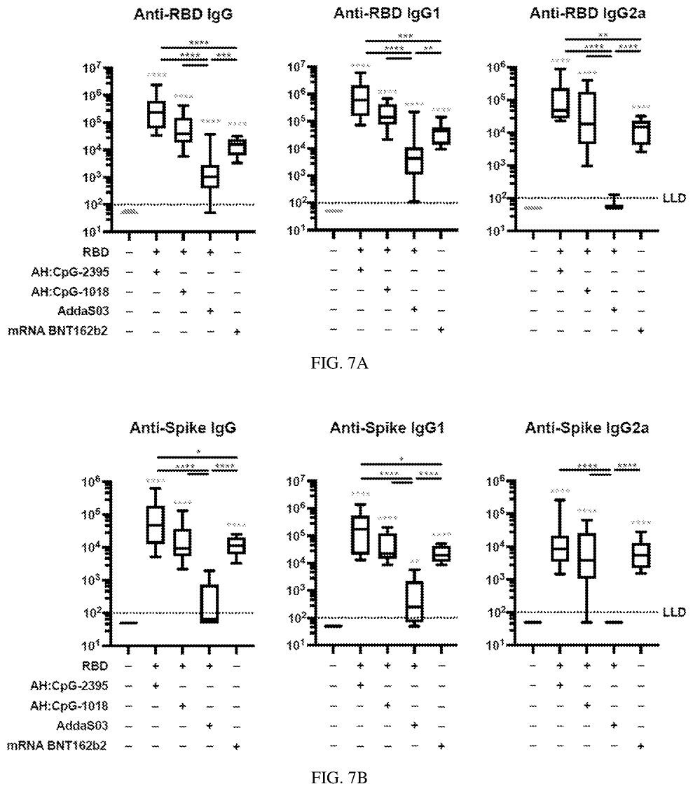

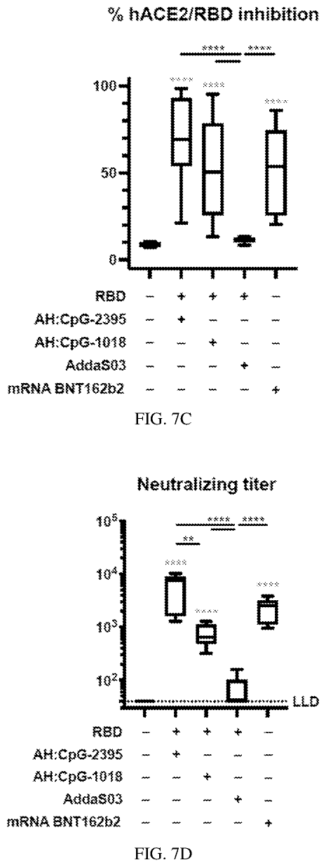

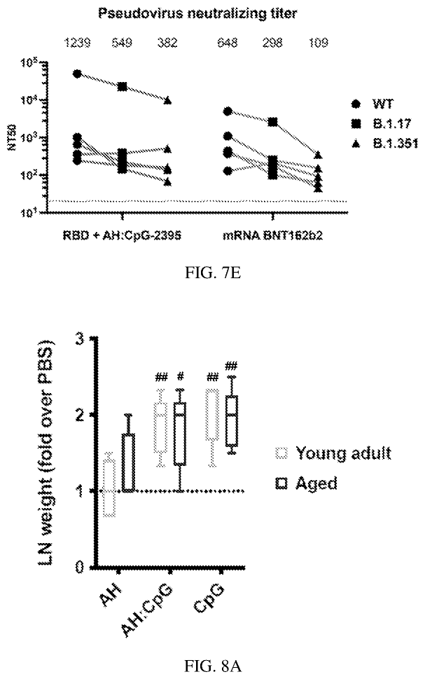

Recently, it has been reported that SARS-CoV-2 mRNA vaccines are more immunogenic than RBD adjuvanted with oil-in-water emulsions³⁶. To assess whether this is a general feature of RBD protein vaccines, the clinical-grade authorized BNT162b2 Spike protein mRNA vaccine (Pfizer-BioNTech) was used as a benchmark and compared to RBD formulated with AddaS03 (a commercially available version of the oil-in-water emulsion AS03) or AH:CpG in aged mice. Along with CpG-2395, CpG-1018 was also tested. CpG-1018 is included in the Heplisav vaccine and has also been tested in combination Spike and AH in studies including a phase 1 clinical trial^{12,16,37}. In accordance with previously published data, the mRNA was highly immunogenic, while RBD formulated with AddaS03 failed to induce significant levels of neutralizing antibodies (FIGS. 7A-7D). Of note, both AH:CpG formulations elicited levels of anti-RBD (FIG. 7A), anti-Spike (FIG. 7B) and neutralizing Abs (FIGS. 7C-7D) comparable to the mRNA vaccine. SARS-CoV-2 variants such as B.1.1.7 and B.1.351 have emerged with reduced neutralization from serum samples of convalescent or vaccinated individuals³⁸⁻⁴¹. The mRNA BNT162b2 vaccine has been reported to maintain its effectiveness against severe COVID-19 occurring from the B.1.351 variant at greater than 90%⁴². It was therefore evaluated whether RBD+AH:CpG elicits neutralizing antibodies against these variants similarly to formulations with BNT162b2 mRNA. As expected, antibody titers against these variants were reduced, especially against the B.1.351 (FIG. 7E). The neutralization titers of RBD+AH:CpG decreased by 3.2-fold against B.1.351, and the mRNA BNT 162b2 decreased by 6.0-fold. However, neutralizing titers against the B.1.351 are similar between RBD+AH:CpG and mRNA BNT162b2 (FIG. 7E; see geometric mean titer (GMT) 382 vs 109, respectively).

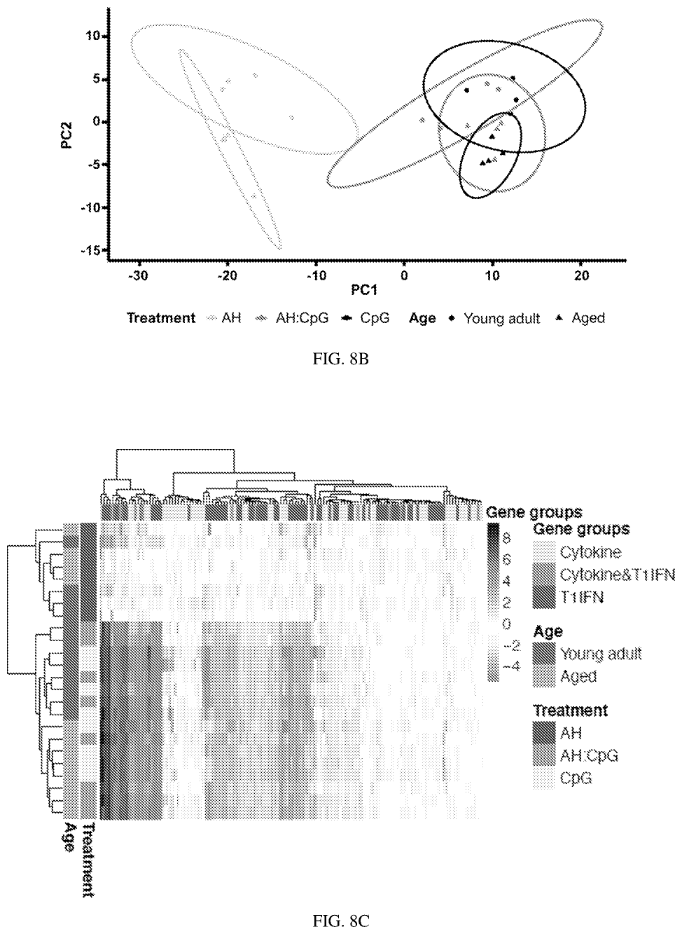

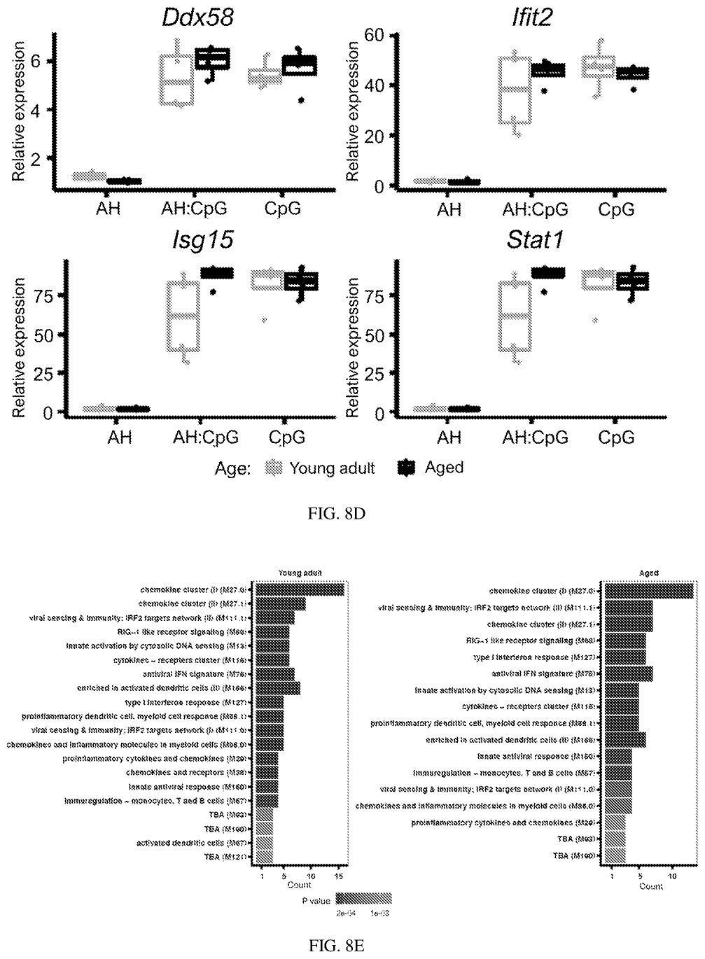

Lymph nodes (LNs) are critical sites for the interaction between innate and adaptive immune systems and orchestrate the development of vaccine immune responses^{43,44}. Of note, activation of the innate immune system can induce a rapid response in the LN characterized by LN expansion driven by lymphocyte accrual and expression of pro-inflammatory molecules^{45,46}. To gain further insights into the mechanism of action of the AH:CpG formulation, draining LNs (dLNs) were collected 24 hours post injection of AH:CpG, or either adjuvant alone. CpG and AH:CpG induced comparable dLNs expansion in both age groups (FIG. 8A). To further characterize the molecular events associated with these treatments, RNA isolated from dLNs after injection of vehicle, CpG, or AH:CpG was subjected to quantitative real-time PCR array comprised of 157 genes related to cytokines, chemokines, and type 1 IFN responses. Principal component analysis and hierarchical cluster analysis demonstrated marked separation between AH and CpG-containing treatments, whereas similar patterns were observed between groups treated with AH:CpG and CpG alone, in both age groups (FIGS. 8B-8C). Generalized linear model analysis comparing gene expressions after AH, CpG, and AH:CpG treatments further revealed similar gene enrichment pattern between young adult and aged mice (FIGS. 8D-8E). These results suggest the CpG and AH:CpG activate similar pathways in young and aged mice to elicit a LN innate response.

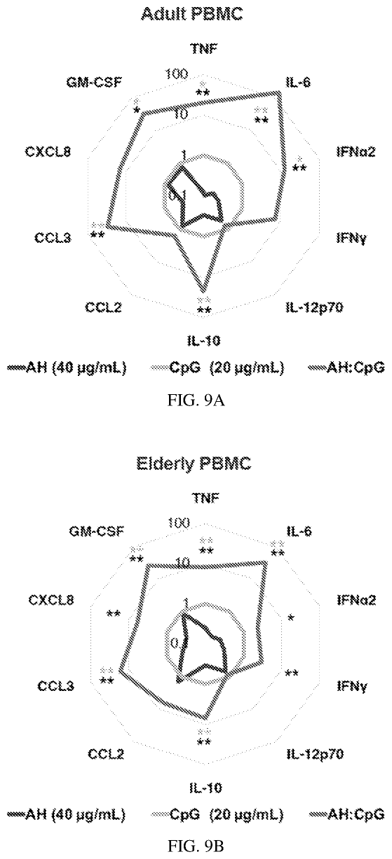

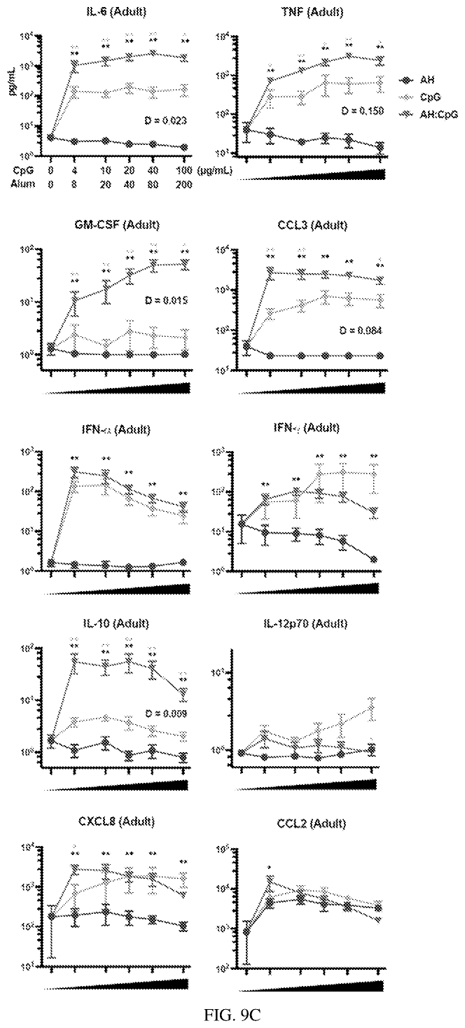

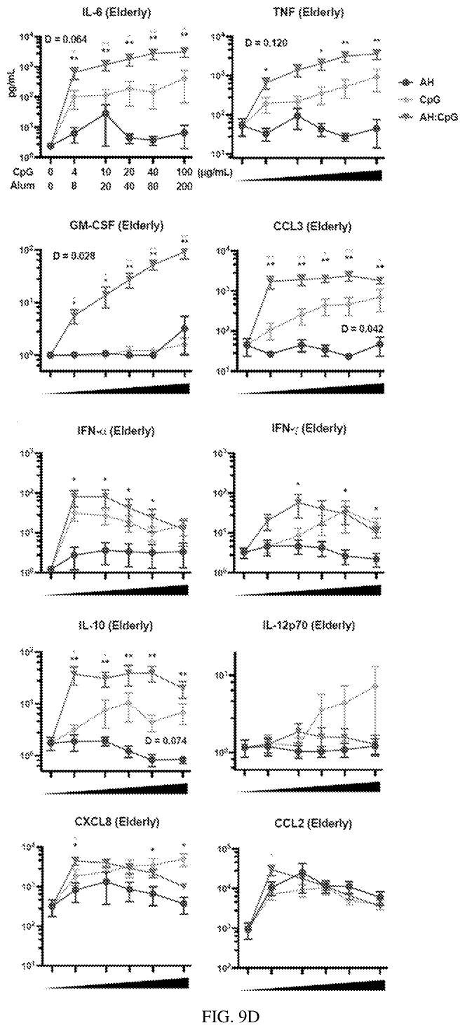

In order to assess the translational relevance of an adjuvant formulation it is key to confirm its ability to activate human immune cells. To this end, human peripheral blood mononuclear cells (PBMCs) isolated from young adults (18-40 years old) and elderly adults (≥65 years old) were stimulated with CpG, AH, and the admixed formulation, and cytokine and chemokine production was measured. Whereas AH induced limited or no cytokine production, both CpG alone and AH:CpG activated young adult and elderly PBMCs in a concentration-dependent manner (FIGS. 9A-9D). PBMCs of both age groups treated with AH:CpG induced significantly higher production of various proinflammatory cytokines and chemokines than those treated with CpG alone. Of note, CpG and AH synergistically, as defined mathematically (D value), induced IL-6, IL-10, TNF, CCL3, and GM-CSF production in both young adult and elderly PBMCs (FIGS. 9C-9D).

These data show that various AH:PRR-A formulation can induce potent anti-RBD responses in both young and aged mice, overcoming both poor immunogenicity of the antigen and impaired immune response of the aged. Unique immunological properties of the AH:CpG adjuvant formulation have been demonstrated, as has synergistic enhancement of production of multiple cytokines and chemokines from human adult and elderly PBMCs in vitro. These data indicate that formulating RBD with AH:PRR-A, such as CpG, represents a promising approach to developing practical (e.g., not requiring freezing), scalable, and affordable vaccines that may be effective across multiple age groups and could potentially include multiple RBD proteins to achieve cross-strain protection.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### REFERENCES

1. Koff, W. C. et al. Development and deployment of COVID-19 vaccines for those most vulnerable. Science Translational Medicine 13, eabd1525, doi:10.1126/scitranslmed.abd1525 (2021).
2. Baden, L. R. et al. Efficacy and Safety of the mRNA-1273 SARS-CoV-2 Vaccine. The New England journal of medicine, doi:10.1056/NEJMoa2035389 (2020).
3. Polack, F. P. et al. Safety and Efficacy of the BNT162b2 mRNA Covid-19 Vaccine. The New England journal of medicine 383, 2603-2615, doi:10.1056/NEJMoa2034577 (2020).
4. Sadoff, J. et al. Interim Results of a Phase 1-2a Trial of Ad26.COV2.S Covid-19 Vaccine. The New England journal of medicine, doi:10.1056/NEJMoa2034201 (2021).
5. Corbett, K. S. et al. SARS-CoV-2 mRNA vaccine design enabled by prototype pathogen preparedness. Nature 586, 567-571, doi:10.1038/s41586-020-2622-0 (2020).
6. Corbett, K. S. et al. Evaluation of the mRNA-1273 Vaccine against SARS-CoV-2 in Nonhuman Primates. The New England journal of medicine 383, 1544-1555, doi:10.1056/NEJMoa2024671 (2020).
7. Vogel, A. B. et al. BNT162b vaccines protect rhesus macaques from SARS-CoV-2. Nature 592, 283-289, doi:10.1038/s41586-021-03275-y (2021).
8. Mercado, N. B. et al. Single-shot Ad26 vaccine protects against SARS-CoV-2 in rhesus macaques. Nature 586, 583-588, doi:10.1038/s41586-020-2607-z (2020).
9. Gebre, M. S. et al. Novel approaches for vaccine development. Cell 184, 1589-1603, doi:10.1016/j.cell.2021.02.030 (2021).
10. Katz, I. T., Weintraub, R., Bekker, L. G. & Brandt, A. M. From Vaccine Nationalism to Vaccine Equity—Finding a Path Forward. The New England journal of medicine 384, 1281-1283, doi:10.1056/NEJMp2103614 (2021).
11. Keech, C. et al. Phase 1-2 Trial of a SARS-CoV-2 Recombinant Spike Protein Nanoparticle Vaccine. The New England journal of medicine 383, 2320-2332, doi:10.1056/NEJMoa2026920 (2020).
12. Richmond, P. et al. Safety and immunogenicity of S-Trimer (SCB-2019), a protein subunit vaccine candidate for COVID-19 in healthy adults: a phase 1, randomised, double-blind, placebo-controlled trial. Lancet (London, England), doi:10.1016/s0140-6736(21)00241-5 (2021).
13. Xia, S. et al. Safety and immunogenicity of an inactivated SARS-CoV-2 vaccine, BBIBP-CorV: a randomised, double-blind, placebo-controlled, phase 1/2 trial. Lancet Infect Dis 21, 39-51, doi:10.1016/s1473-3099(20)30831-8 (2021).
14. Zhang, Y. et al. Safety, tolerability, and immunogenicity of an inactivated SARS-CoV-2 vaccine in healthy adults aged 18-59 years: a randomised, double-blind, placebo-controlled, phase 1/2 clinical trial. Lancet Infect Dis 21, 181-192, doi:10.1016/s1473-3099(20)30843-4 (2021).
15. Kyriakidis, N. C., López-Cortés, A., González, E. V., Grimaldos, A. B. & Prado, E. O. SARS-CoV-2 vaccines strategies: a comprehensive review of phase 3 candidates. NPJ vaccines 6, 28, doi:10.1038/s41541-021-00292-w (2021).
16. CTRI. Biological E's novel Covid-19 vaccine of SARS-CoV-2 for protection against Covid-19 disease, <http://ctri.nic.in/Clinicaltrials/pmaindet2.php?trialid=48329&EncHid=&userName=covid-19%20vaccine> (2020).).
17. Coronavirus Vaccine Tracker, <https://www.nytimes.com/interactive/2020/science/coronavirus-vaccine-tracker.html>
18. Yang, J. et al. A vaccine targeting the RBD of the S protein of SARS-CoV-2 induces protective immunity. Nature 586, 572-577, doi:10.1038/s41586-020-2599-8 (2020).
19. Piccoli, L. et al. Mapping Neutralizing and Immunodominant Sites on the SARS-CoV-2 Spike Receptor-Binding Domain by Structure-Guided High-Resolution Serology. Cell 183, 1024-1042.e1021, doi:10.1016/j.cell.2020.09.037 (2020).
20. Premkumar, L. et al. The receptor binding domain of the viral spike protein is an immunodominant and highly specific target of antibodies in SARS-CoV-2 patients. Sci Immunol 5, doi:10.1126/sciimmunol.abc8413 (2020).
21. Esposito, D. et al. Optimizing high-yield production of SARS-CoV-2 soluble spike trimers for serology assays. Protein Expr Purif 174, 105686, doi:10.1016/j.pep.2020.105686 (2020).
22. Dalvie, N. C. et al. Engineered SARS-CoV-2 receptor binding domain improves immunogenicity in mice and elicits protective immunity in hamsters. bioRxiv, doi:10.1101/2021.03.03.433558 (2021).
23. Yang, S. et al. Safety and immunogenicity of a recombinant tandem-repeat dimeric RBD-based protein subunit vaccine (ZF2001) against COVID-19 in adults: two randomised, double-blind, placebo-controlled, phase 1 and 2 trials. Lancet Infect Dis, doi:10.1016/s1473-3099(21)00127-4 (2021).
24. Dai, L. et al. A Universal Design of Betacoronavirus Vaccines against COVID-19, MERS, and SARS. Cell 182, 722-733.e711, doi:10.1016/j.cell.2020.06.035 (2020).
25. Hauser, B. M. et al. Engineered receptor binding domain immunogens elicit pan-coronavirus neutralizing antibodies. bioRxiv, doi:10.1101/2020.12.07.415216 (2020).
26. Tan, T. K. et al. A COVID-19 vaccine candidate using SpyCatcher multimerization of the SARS-CoV-2 spike protein receptor-binding domain induces potent neutralising antibody responses. Nature communications 12, 542, doi:10.1038/s41467-020-20654-7 (2021).
27. Walls, A. C. et al. Elicitation of Potent Neutralizing Antibody Responses by Designed Protein Nanoparticle Vaccines for SARS-CoV-2. Cell 183, 1367-1382.e1317, doi:10.1016/j.cell.2020.10.043 (2020).
28. Yang, L. et al. A recombinant receptor-binding domain in trimeric form generates completely protective immunity against SARS-CoV-2 infection in nonhuman primates. bioRxiv, 2021.2003.2030.437647, doi:10.1101/2021.03.30.437647 (2021).
29. He, L. et al. Single-component, self-assembling, protein nanoparticles presenting the receptor binding domain and stabilized spike as SARS-CoV-2 vaccine candidates. Sci Adv 7, doi:10.1126/sciadv.abf1591 (2021).
30. Reed, S. G., Tomai, M. & Gale, M. J., Jr. New horizons in adjuvants for vaccine development. Curr Opin Immunol 65, 97-101, doi:10.1016/j.coi.2020.08.008 (2020).
31. Pulendran, B., P, S. A. & O'Hagan, D. T. Emerging concepts in the science of vaccine adjuvants. Nature reviews. Drug discovery, 1-22, doi:10.1038/s41573-021-00163-y (2021).
32. O'Hagan, D. T., Lodaya, R. N. & Lofano, G. The continued advance of vaccine adjuvants—‘we can work it out’. Seminars in immunology 50, 101426, doi:10.1016/j.smim.2020.101426 (2020).
33. Lal, H. et al. Efficacy of an adjuvanted herpes zoster subunit vaccine in older adults. The New England journal of medicine 372, 2087-2096, doi:10.1056/NEJMoa1501184 (2015).
34. Cunningham, A. L. et al. Efficacy of the Herpes Zoster Subunit Vaccine in Adults 70 Years of Age or Older. The New England journal of medicine 375, 1019-1032, doi:10.1056/NEJMoa1603800 (2016).
35. Leist, S. R. et al. A Mouse-Adapted SARS-CoV-2 Induces Acute Lung Injury and Mortality in Standard Laboratory Mice. Cell 183, 1070-1085.e1012, doi:10.1016/j.cell.2020.09.050 (2020).
36. Lederer, K. et al. SARS-CoV-2 mRNA Vaccines Foster Potent Antigen-Specific Germinal Center Responses Associated with Neutralizing Antibody Generation. Immunity 53, 1281-1295.e1285, doi:10.1016/j.immuni.2020.11.009 (2020).
37. Liang, J. G. et al. S-Trimer, a COVID-19 subunit vaccine candidate, induces protective immunity in nonhuman primates. Nature communications 12, 1346, doi:10.1038/s41467-021-21634-1 (2021).
38. Garcia-Beltran, W. F. et al. Multiple SARS-CoV-2 variants escape neutralization by vaccine-induced humoral immunity. Cell, doi:10.1016/j.cell.2021.03.013 (2021).
39. Kuzmina, A. et al. SARS-CoV-2 spike variants exhibit differential infectivity and neutralization resistance to convalescent or post-vaccination sera. Cell host & microbe, doi:10.1016/j.chom.2021.03.008 (2021).
40. Shen, X. et al. Neutralization of SARS-CoV-2 Variants B.1.429 and B.1.351. The New England journal of medicine, doi:10.1056/NEJMc2103740 (2021).
41. Wang, Z. et al. mRNA vaccine-elicited antibodies to SARS-CoV-2 and circulating variants. Nature 592, 616-622, doi:10.1038/s41586-021-03324-6 (2021).
42. Abu-Raddad, L. J., Chemaitelly, H. & Butt, A. A. Effectiveness of the BNT162b2 Covid-19 Vaccine against the B.1.1.7 and B.1.351 Variants. The New England journal of medicine, doi:10.1056/NEJMc2104974 (2021).
43. Iwasaki, A. & Medzhitov, R. Control of adaptive immunity by the innate immune system. Nat Immunol 16, 343-353, doi:10.1038/ni.3123 (2015).
44. Pollard, A. J. & Bijker, E. M. A guide to vaccinology: from basic principles to new developments. Nature reviews. Immunology 21, 83-100, doi:10.1038/s41577-020-00479-7 (2021).
45. Grant, S. M., Lou, M., Yao, L., Germain, R. N. & Radtke, A. J. The lymph node at a glance—how spatial organization optimizes the immune response. J Cell Sci 133, doi:10.1242/jcs.241828 (2020).
46. Acton, S. E. & Reis e Sousa, C. Dendritic cells in remodeling of lymph nodes during immune responses. Immunol Rev 271, 221-229, doi:10.1111/imr.12414 (2016).
47. Williamson, E. J. et al. Factors associated with COVID-19-related death using OpenSAFELY. Nature 584, 430-436, doi:10.1038/s41586-020-2521-4 (2020).
48. Centers for Disease Control and Prevention (CDC). COVID-19 Hospitalization and Death by Age, <https://www.cdc.gov/coronavirus/2019-ncov/covid-data/investigations-discovery/hospitalization-death-by-age.html>(
49. Hotez, P. J. & Bottazzi, M. E. Developing a low-cost and accessible COVID-19 vaccine for global health. PLoS Negl Trop Dis 14, e0008548, doi:10.1371/journal.pntd.0008548 (2020).
50. McMahan, K. et al. Correlates of protection against SARS-CoV-2 in rhesus macaques. Nature, doi:10.1038/s41586-020-03041-6 (2020).
51. Arunachalam, P. S. et al. Adjuvanting a subunit SARS-CoV-2 nanoparticle vaccine to induce protective immunity in non-human primates. bioRxiv, 2021.2002.2010.430696, doi:10.1101/2021.02.10.430696 (2021).
52. Chen, W. H. et al. Genetic modification to design a stable yeast-expressed recombinant SARS-CoV-2 receptor binding domain as a COVID-19 vaccine candidate. Biochim Biophys Acta Gen Subj 1865, 129893, doi:10.1016/j.bbagen.2021.129893 (2021).
53. Chen, W. H. et al. Yeast-expressed SARS-CoV recombinant receptor-binding domain (RBD219-N1) formulated with aluminum hydroxide induces protective immunity and reduces immune enhancement. Vaccine 38, 7533-7541, doi:10.1016/j.vaccine.2020.09.061 (2020).
54. Pollet, J. et al. SARS-CoV-2 RBD219-N1C1: A yeast-expressed SARS-CoV-2 recombinant receptor-binding domain candidate vaccine stimulates virus neutralizing antibodies and T-cell immunity in mice. Human vaccines & immunotherapeutics, 1-11, doi:10.1080/21645515.2021.1901545 (2021).
55. Paavonen, J. et al. Efficacy of human papillomavirus (HPV)-16/18 AS04-adjuvanted vaccine against cervical infection and precancer caused by oncogenic HPV types (PATRICIA): final analysis of a double-blind, randomised study in young women. Lancet (London, England) 374, 301-314, doi:10.1016/s0140-6736(09)61248-4 (2009).
56. Kayraklioglu, N., Horuluoglu, B. & Klinman, D. M. CpG Oligonucleotides as Vaccine Adjuvants. Methods in molecular biology (Clifton, N.J.) 2197, 51-85, doi:10.1007/978-1-0716-0872-2_4 (2021).
57. Campbell, J. D. Development of the CpG Adjuvant 1018: A Case Study. Methods in molecular biology (Clifton, N.J.) 1494, 15-27, doi:10.1007/978-1-4939-6445-1_2 (2017).
58. Cooper, C. L. et al. CPG 7909, an immunostimulatory TLR9 agonist oligodeoxynucleotide, as adjuvant to Engerix-B HBV vaccine in healthy adults: a double-blind phase I/II study. J Clin Immunol 24, 693-701, doi:10.1007/s10875-004-6244-3 (2004).
59. Maletto, B., Rópolo, A., Morón, V. & Pistoresi-Palencia, M. C. CpG-DNA stimulates cellular and humoral immunity and promotes Th1 differentiation in aged BALB/c mice. Journal of leukocyte biology 72, 447-454 (2002).
60. Maletto, B. A. et al. CpG oligodeoxinucleotides functions as an effective adjuvant in aged BALB/c mice. Clinical immunology (Orlando, Fla.) 117, 251-261, doi:10.1016/j.clim.2005.08.016 (2005).
61. Manning, B. M., Enioutina, E. Y., Visic, D. M., Knudson, A. D. & Daynes, R. A. CpG DNA functions as an effective adjuvant for the induction of immune responses in aged mice. Exp Gerontol 37, 107-126, doi:10.1016/s0531-5565(01)00157-7 (2001).
62. Ming, F. et al. Immunization of aged pigs with attenuated pseudorabies virus vaccine combined with CpG oligodeoxynucleotide restores defective Th1 immune responses. PloS one 8, e65536, doi:10.1371/journal.pone.0065536 (2013).
63. Qin, W. et al. CpG ODN enhances immunization effects of hepatitis B vaccine in aged mice. Cell Mol Immunol 1, 148-152 (2004).
64. Sen, G., Chen, Q. & Snapper, C. M. Immunization of aged mice with a pneumococcal conjugate vaccine combined with an unmethylated CpG-containing oligodeoxynucleotide restores defective immunoglobulin G antipolysaccharide responses and specific CD4+-T-cell priming to young adult levels. Infection and immunity 74, 2177-2186, doi:10.1128/iai.74.4.2177-2186.2006 (2006).
65 Sablan, B. P. et al. Demonstration of safety and enhanced seroprotection against hepatitis B with investigational HBsAg-1018 ISS vaccine compared to a licensed hepatitis B vaccine. Vaccine 30, 2689-2696, doi:10.1016/j.vaccine.2012.02.001 (2012).
66. Janssen, J. M., Jackson, S., Heyward, W. L. & Janssen, R. S. Immunogenicity of an investigational hepatitis B vaccine with a toll-like receptor 9 agonist adjuvant (HBsAg-1018) compared with a licensed hepatitis B vaccine in subpopulations of healthy adults 18-70 years of age. Vaccine 33, 3614-3618, doi:10.1016/j.vaccine.2015.05.070 (2015).
67. Kuo, T. Y. et al. Development of CpG-adjuvanted stable prefusion SARS-CoV-2 spike antigen as a subunit vaccine against COVID-19. Sci Rep 10, 20085, doi:10.1038/s41598-020-77077-z (2020).
68. Lien, C.-E. et al. CpG-adjuvanted stable prefusion SARS-CoV-2 spike protein protected hamsters from SARS-CoV-2 challenge. bioRxiv, 2021.2001.2007.425674, doi:10.1101/2021.01.07.425674 (2021).
69. Dowling, D. J. et al. TLR7/8 adjuvant overcomes newborn hyporesponsiveness to pneumococcal conjugate vaccine at birth. JCI insight 2, e91020, doi:10.1172/jci.insight.91020 (2017).
70 Garrido, C. et al. SARS-CoV-2 Vaccines Elicit Durable Immune Responses in Infant Rhesus Macaques. bioRxiv, 2021.2004.2005.438479, doi:10.1101/2021.04.05.438479 (2021).
71. Wu, K. et al. Variant SARS-CoV-2 mRNA vaccines confer broad neutralization as primary or booster series in mice. bioRxiv, 2021.2004.2013.439482, doi:10.1101/2021.04.13.439482 (2021).
72. Baylor, N. W., Egan, W. & Richman, P. Aluminum salts in vaccines—US perspective. Vaccine 20 Suppl 3, S18-23, doi:10.1016/s0264-410x(02)00166-4 (2002).
73. Borriello, F. et al. Identification and Characterization of Stimulator of Interferon Genes As a Robust Adjuvant Target for Early Life Immunization. Front Immunol 8, 1772, doi:10.3389/fimmu.2017.01772 (2017).
74. Tan, C. W. et al. A SARS-CoV-2 surrogate virus neutralization test based on antibody-mediated blockage of ACE2-spike protein-protein interaction. Nat Biotechnol 38, 1073-1078, doi:10.1038/s41587-020-0631-z (2020).
75. Yu, J. et al. DNA vaccine protection against SARS-CoV-2 in rhesus macaques. Science 369, 806-811, doi:10.1126/science.abc6284 (2020).
76. Yu, J. et al. Deletion of the SARS-CoV-2 Spike Cytoplasmic Tail Increases Infectivity in Pseudovirus Neutralization Assays. Journal of Virology, JVI.00044-00021, doi:10.1128/jvi.00044-21 (2021).
77. van Haren, S. D. et al. Age-Specific Adjuvant Synergy: Dual TLR7/8 and Mincle Activation of Human Newborn Dendritic Cells Enables Th1 Polarization. Journal of immunology (Baltimore, Md.: 1950) 197, 4413-4424, doi:10.4049/jimmunol.1600282 (2016).
78. Berenbaum, M. C. Correlations between methods for measurement of synergy. The Journal of infectious diseases 142, 476-480, doi:10.1093/infdis/142.3.476 (1980).

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of inducing an immune response to a Beta coronavirus in a subject in need thereof, the method comprising administering to the subject a Beta coronavirus antigen and an adjuvantation system comprising a pattern recognition receptors (PRR) agonist.

2. The method of claim 1, wherein the PRR agonist is a Toll-like receptor (TLR) 3 agonist, a TLR4 agonist, a TLR9 agonist, or a Stimulator of Interferon Genes (STING) agonist.

3. The method of claim 2, wherein the TLR3 agonist comprises polyinosinic:polycytidylic acid (Poly I:C).

4. The method of claim 2, wherein the TLR4 agonist comprises phosphorylated hexa-acyl disaccharide (PHAD).

5. The method of claim 2, wherein the TLR9 agonist comprises a CpG-containing oligodeoxynucleotide (CpG-ODN).

6. The method of claim 5, wherein the CpG-containing oligodeoxynucleotide is a class A CpG-ODN, a class B CpG-ODN, or a class C CpG-ODN.

7. The method of claim 6, wherein the class B CpG-ODN is CpG-ODN-1018.

8. The method of claim 6, wherein the class C CpG-ODN is CpG-ODN-2395.

9. The method of claim 2, wherein the STING agonist comprises 2′3′-cGAMP.

10. The method of any one of claims 1-9, wherein the adjuvantation system further comprises alum.

11. The method of claim 10, wherein the PRR agonist is adsorbed into the alum.

12. The method of any one of claims 1-11, wherein the Beta coronavirus is selected from Middle East Respiratory Syndrome coronavirus (MERS-CoV), Severe Acute Respiratory Syndrome (SARS)-associated coronavirus (SARS-CoV)-1, and SARS-CoV-2.

13. The method of any one of claims 1-12, wherein the Beta coronavirus antigen comprises a Beta coronavirus protein or polypeptide.

14. The method of any one of claims 1-12, wherein the antigen comprises a nucleic acid encoding a Beta coronavirus protein or a polypeptide.

15. The method of claim 14, wherein the nucleic acid is DNA or RNA.

16. The method of claim 15, wherein the RNA is a messenger RNA (mRNA).

17. The method of any one of claims 13-16, wherein the Beta coronavirus protein or polypeptide comprises a Beta coronavirus spike protein or spike protein receptor binding domain.

18. The method of claim 17, wherein the Beta coronavirus spike protein is a MERS-CoV spike protein, SARS-CoV-1 spike protein, or SARS-CoV-2 spike protein.

19. The method of any one of claims 1-12, wherein the antigen comprises a viral particle of MERS-CoV, SARS-CoV-1, or SARS-CoV-2.

20. The method of any one of claims 1-12, wherein the antigen comprises killed or inactivated MERS-CoV, SARS-CoV-1, or SARS-CoV-2.

21. The method of any one of claims 1-12, wherein the antigen comprises killed or live attenuated MERS-CoV, SARS-CoV-1, or SARS-CoV-2.

22. The method of any one of claims 1-21, wherein the subject is human.

23. The method of claim 22, wherein the subject is a human neonate, an infant, an adult, or an elderly.

24. The method of claim 13, wherein the subject is an elderly human.

25. The method of any one of claims 1-21, wherein the subject is a companion animal or a research animal.

26. The method of any one of claims 1-25, wherein the subject is immune-compromised, has chronic lung disease, asthma, cardiovascular disease, cancer, obesity, diabetes, chronic kidney disease, and/or liver disease.

27. The method of any one of claims 1-26, wherein the Beta coronavirus antigen and the adjuvantation system are administered simultaneously.

28. The method of any one of claims 1-26, wherein the antigen and the adjuvantation system are administered separately.

29. The method of any one of claims 1-28, wherein the administering is done intramuscularly, intradermally, orally, intravenously, topically, intranasally, or sublingually.

30. The method of any one of claims 1-29, wherein the administration is prophylactic.

31. The method of any one of claims 1-30, wherein the adjuvantation system enhances B cell immunity.

32. The method of any one of claims 1-31, wherein the adjuvantation system enhances the production of antigen-specific antibodies, compared to when the Beta coronavirus antigen is administered alone.

33. The method of claim 32, wherein the antigen-specific antibodies are immunoglobulin G (IgG).

34. The method of claim 33, wherein the antigen-specific antibodies are subclass 1 IgG (IgG1) or subclass 2 IgG (IgG2).

35. The method of any one of claims 32-34, wherein the antigen-specific antibodies are neutralizing antibodies against a variant of SARS-CoV-2.

36. The method of claim 35, wherein the variant of SARS-CoV-2 is wild-type SARS-CoV-2, B.1.1.7 SARS-CoV-2, or B.1.351 SARS-CoV-2.

37. The method of any one of claims 1-36, wherein the adjuvantation system enhances the cytokine production of peripheral blood mononuclear cells (PBMCs), compared to when the Beta coronavirus antigen is administered alone.

38. The method of claim 37, wherein the PBMCs are antigen-specific T cells.

39. The method of claim 37 or claim 38, wherein the adjuvantation system enhances the cytokine production of IL-2, IL-6, IL-10, TNF, IFNα, IFNγ, CCL3, CXCL8 and/or GM-CSF.

40. The method of any one of claims 1-39, wherein the adjuvantation system polarizes the innate immune response toward T follicular helper (Tfh) cell immunity.

41. The method of any one of claims 1-40, wherein the adjuvantation system polarizes the innate immune response toward T helper 1 (Th1) cell immunity.

42. The method of any one of claims 1-41, wherein the adjuvantation system enhances the inhibition of interaction between angiotensin-converting enzyme 2 (ACE2) and Beta coronavirus spike protein, compared to when the Beta coronavirus antigen is administered alone.

43. The method of any one of claims 1-42, wherein the adjuvantation system prolongs a protective effect in the subject against the Beta coronavirus antigen, compared to when the Beta coronavirus antigen is administered alone.

44. The method of any one of claims 1-43, wherein the adjuvantation system increases rate of an immune response, compared to when the Beta coronavirus antigen is administered alone.

45. The method of any one of claims 1-44, wherein the Beta coronavirus antigen produces a same level of immune response against the antigen at a lower dose in the presence of the adjuvantation system, compared to when the Beta coronavirus antigen is administered alone.

46. The method of any one of claims 1-45, wherein the likelihood of antibody disease enhancement (ADE) is reduced in the subject, compared to when the Beta coronavirus antigen is administered alone.

47. An adjuvantation system comprising a pattern recognition receptor (PRR) agonist for use in inducing an immune response against a Beta coronavirus in a subject in need thereof.

48. The adjuvantation system of claim 47, wherein the PRR agonist is a TLR3 agonist, a TLR4 agonist, a TLR9 agonist, or a STING agonist.

49. The adjuvantation system of claim 48, wherein the TLR3 agonist comprises polyinosinic:polycytidylic acid (Poly I:C).

50. The adjuvantation system of claim 48, wherein the TLR4 agonist comprises phosphorylated hexa-acyl disaccharide (PHAD).

51. The adjuvantation system of claim 48, wherein the TLR9 agonist comprises a CpG-containing oligodeoxynucleotide (CpG-ODN).

52. The adjuvantation system of claim 51, wherein the CpG-containing oligodeoxynucleotide is a class A CpG-ODN, a class B CpG-ODN, or a class C CpG-ODN.

53. The adjuvantation system of claim 52, wherein the class B CpG-ODN is CpG-ODN-1018.

54. The adjuvantation system of claim 52, wherein the class C CpG-ODN is CpG-ODN-2395.

55. The adjuvantation system of claim 48, wherein the STING agonist comprises 2′3′-cGAMP.

56. An adjuvantation system comprising a pattern recognition receptor (PRR) agonist and alum for use in inducing an immune response against a Beta coronavirus in a subject in need thereof.

57. The adjuvantation system of claim 56, wherein the PRR agonist is a TLR3 agonist, a TLR4 agonist, a TLR9 agonist, or a STING agonist.

58. The adjuvantation system of claim 57, wherein the TLR3 agonist comprises polyinosinic:polycytidylic acid (Poly I:C).

59. The adjuvantation system of claim 57, wherein the TLR4 agonist comprises phosphorylated hexa-acyl disaccharide (PHAD).

60. The adjuvantation system of claim 57, wherein the TLR9 agonist comprises a CpG-containing oligodeoxynucleotide (CpG-ODN).

61. The adjuvantation system of claim 60, wherein the CpG-containing oligodeoxynucleotide is a class A CpG-ODN, a class B CpG-ODN, or a class C CpG-ODN.

62. The adjuvantation system of claim 61, wherein the class B CpG-ODN is CpG-ODN-1018.

63. The adjuvantation system of claim 61, wherein the class C CpG-ODN is CpG-ODN-2395.

64. The adjuvantation system of claim 57, wherein the STING agonist comprises 2′3′-cGAMP.

65. An immunogenic composition comprising a Beta coronavirus antigen and an adjuvantation system comprising a pattern recognition receptor (PRR) agonist.

66. The immunogenic composition of claim 65, wherein the PRR agonist is a TLR3 agonist, a TLR4 agonist, a TLR9 agonist, or a STING agonist.

67. The immunogenic composition of claim 66, wherein the TLR3 agonist comprises polyinosinic:polycytidylic acid (Poly I:C).

68. The immunogenic composition of claim 66, wherein the TLR4 agonist comprises phosphorylated hexa-acyl disaccharide (PHAD).

69. The immunogenic composition of claim 66, wherein the TLR9 agonist comprises a CpG-containing oligodeoxynucleotide (CpG-ODN).

70. The immunogenic composition of claim 69, wherein the CpG-containing oligodeoxynucleotide is a class A CpG-ODN, a class B CpG-ODN, or a class C CpG-ODN.

71. The immunogenic composition of claim 70, wherein the class B CpG-ODN is CpG-ODN-1018.

72. The immunogenic composition of claim 70, wherein the class C CpG-ODN is CpG-ODN-2395.

73. The immunogenic composition of claim 66, wherein the STING ligand comprises 2′3′-cGAMP.

74. The immunogenic composition of any one of claims 65-73, wherein the adjuvantation system further comprises alum.

75. The immunogenic composition of claim 74, wherein the PRR agonist is adsorbed into the alum.

76. The immunogenic composition of any one of claims 65-75, wherein Beta coronavirus is selected from Middle East Respiratory Syndrome coronavirus (MERS-CoV), Severe Acute Respiratory Syndrome (SARS)-associated coronavirus (SARS-CoV)-1, and SARS-CoV-2.

77. The immunogenic composition of any one of claims 65-76, wherein the Beta coronavirus antigen comprises a Beta coronavirus protein or polypeptide.

78. The immunogenic composition of any one of claims 65-76, wherein the antigen comprises a nucleic acid encoding a Beta coronavirus protein or a polypeptide.

79. The immunogenic composition of claim 78, wherein the nucleic acid is DNA or RNA.

80. The immunogenic composition of claim 79, wherein the RNA is a messenger RNA (mRNA).

81. The immunogenic composition of any one of claims 77-80, wherein the Beta coronavirus protein or polypeptide comprises a Beta coronavirus spike protein or spike protein receptor binding domain.

82. The immunogenic composition of claim 81, wherein the Beta coronavirus spike protein is a MERS-CoV spike protein, SARS-CoV-1 spike protein, or SARS-CoV-2 spike protein.

83. The immunogenic composition of any one of claims 65-76, wherein the antigen comprises a viral particle of MERS-CoV, SARS-CoV-1, or SARS-CoV-2.

84. The immunogenic composition of any one of claims 65-76, wherein the antigen comprises killed or inactivated MERS-CoV, SARS-CoV-1, or SARS-CoV-2.

85. The immunogenic composition of any one of claims 65-76, wherein the antigen comprises killed or live attenuated MERS-CoV, SARS-CoV-1, or SARS-CoV-2.
